(19) United States
(12) Patent Application Publication
(10) Pub. No.: US 2015/0118231 A1
(43) Pub. Date: Apr. 30, 2015

(54) APOE4 ANTIBODIES FOR TREATMENT OF NEURODEGENERATIVE CONDITIONS

(71) Applicant: RAMOT AT TEL-AVIV UNIVERSITY LTD., Tel-Aviv (IL)

(72) Inventor: Daniel Michaelson, Tel Aviv (IL)

(21) Appl. No.: 14/399,217

(22) PCT Filed: May 7, 2013
(86) PCT No.: PCT/IL2013/050439

§ 371 Date: Nov. 6, 2014

Related U.S. Application Data
(60) Provisional application No. 61/643,917, filed on May 8, 2012.

(51) Int. Cl. C07K 16/18; A61K 39/395; A61K 31/192

(52) U.S. Cl. CPC C07K 16/18; A61K 31/192; A61K 39/3955; C07K 2317/24; C07K 2317/622; A61K 2039/505
USPC 424/135.1; 424/141.1; 424/133.1

(57) ABSTRACT

The present invention relates to methods and compositions for treating neurodegenerative conditions, such as Alzheimer's Disease, by systemic administration of specific apoE4 antibodies.

### FIELD OF THE INVENTION

The present invention relates to compositions and methods for treating neurodegenerative conditions, such as Alzheimer's Disease, by systemic administration of specific apoE4 antibodies.

### BACKGROUND OF THE INVENTION

Alzheimer's Disease (AD), the most prevalent form of dementia in the elderly, is characterized by cognitive decline and by the occurrence of brain senile plaques and neurofibrillary tangles (NFT), as well as synapse and neuronal loss in the brain. The senile plaques contain a 40-42-amino acid-long amyloid-beta (A-beta or Aβ) peptide derived from amyloid precursor protein (APP). Aβ is also present in the brain as soluble oligomers, which play an important and early role in neurodegeneration in AD. Various treatment methods have been suggested to affect the deposition of amyloid-beta (Aβ) peptide. For example, it has been shown that systemic administration of antibodies against amyloid beta, are able to reach the brain. This may be achieved due to the very low concentration of Aβ in circulating serum, as opposed to its very high levels of expression in the brain.

The neurofibrillary tangles (NFT) contain abnormal aggregates of the microtubule-associated protein tau, which lead to disruption of the neuronal cytoskeleton followed by neurodegeneration and death. Several chemical modifications have been described in NFT tau, of which hyperphosphorylation is a key event. Other findings suggest that tau also forms soluble extracellular neurotoxic oligomers. Genetic studies of familial AD revealed that mutations in APP and in two homologous genes, termed PSEN1 and PSEN2, encoding the presenilin (PS) PS1 and PS2 proteins, result in elevated levels of Aβ.

Further genetic studies revealed allelic segregation of the apolipoprotein E (apoE) gene to families with a higher risk of late onset AD and to sporadic AD. There are three major alleles of apoE, termed E2 (apoE2), E3 (apoE3), and E4 (apoE4), of which apoE4 is the AD risk factor. The frequency of apoE4 in sporadic AD is over 50%, and it increases the risk for AD by lowering the age of onset of the disease by 7 to 9 years per allele copy. Interestingly, apoE2 is protective in this regard as it decreases the probability of AD.

Studies with several lines of APP, tau, and apoE4 mice revealed marked effects of environmental conditions on Aβ, tau and synaptic pathology. These findings suggest that the phenotypic expression of the AD-related genotypes is affected by environmental factors.

Various studies have shown that brain atrophy and Aβ deposition begin during the preclinical stage of the disease and can predict future cognitive decline. Synaptic dysfunction and loss is the earliest histological neuronal pathology in AD. Furthermore, it is also apparent in mild cognitively impaired (MCI) individuals prior to their conversion to clinical AD. AD synaptic pathology is associated with early loss of dendritic spines and with presynaptic and post synaptic impairments, which correlate with cognitive decline at the early stages of the disease. Synaptic pathology is more pronounced in distinct brain areas such as the hippocampus and may be mediated by impairments that originate either in the synaptic terminal or in the cell body. The major implication of these findings is that treatment provided during the clinical phase may thus be too late and should be provided to the appropriate at-risk subpopulation of clinically normal subjects beforehand. In view of the impracticality of pursuing such longitudinal studies over decades, the problem may be studied first in animal models.

Pathologically, apoE4 is associated with increased deposition of Aβ, with impaired neuronal plasticity, and with increased neuropathology. Declining memory and brain pathology have been reported in middle-aged and young apoE4 carriers, with ongoing normal clinical status, suggesting that the effects of apoE4 start decades before the onset of AD. ApoE4 is also associated with other neurodegenerative conditions including poor outcome after traumatic brain injury and brain hemorrhage. The mechanisms underlying these pathological effects of apoE4 are not fully understood. Exemplary mechanisms that have been proposed include: (i) Synergistic interactions with the amyloid cascade: The findings that Aβ deposition is specifically elevated in apoE4-positive AD patients and that apoE4 and Aβ interact synergistically in both in vivo and in vitro models, led to the suggestion that the pathological effects of apoE4 are mediated by cross-talk interactions with the amyloid cascade. (ii) ApoE4 and lipids: The central role of apoE in the transport and delivery of brain lipids and the finding that apoE3 and apoE4 interact differentially with lipids led to the proposal that the pathological effects of apoE4 are mediated via lipid-related mechanisms. Accordingly, in vitro studies revealed that apoE4 is less efficient than apoE3 in transporting cholesterol and in promoting cholesterol efflux from neurons and astrocytes. It has recently been shown by the inventor of the present invention and others that the pathological effects of apoE4 in targeted replacement mice are prevented by a fish oil (DHA) diet that was provided immediately following weaning. (iii) Intraneuronal apoE4 and tau: ApoE is expressed in stressed and injured neurons and transgenic over-expression of apoE4 in neurons increases tau phosphorylation. This led to an additional hypothesis, namely, that the pathological effects of apoE4 are mediated by stimulation of tau hyperphosphorylation. This could be mediated by C-terminal-fragments of apoE, which are detected in AD brains and in brain neurons of apoE4 mouse, and which enter the cytosol and interact directly with tau, or by alternative mechanisms. (iv) ApoE receptors and signaling: Recent studies revealed that the apoE isoforms interact differently with apoE receptors, suggesting that the effects of apoE4 may be mediated via modulation of distinct receptor signaling cascades. The existence of several suggested mechanisms has important implications regarding the design of apoE4-directed therapy and the use of appropriate models.

Most of the apoE4 AD carriers are heterozygotes and possess the mixed apoE3/apoE4 genotype. Accordingly, the increased risk for AD in these subjects may be due either to the loss of one copy of the “good” apoE3 allele or to a dominant negative effect of the “bad” apoE4 allele. Various studies, which compared the in vivo effects of apoE4 to those of both apoE3 and apoE-deficient mice, have shown that the effects of apoE4 are associated with a gain of toxic function that is not present in the apoE-deficient mice. However, apoE4 carriers have lower levels of apoE than do the apoE3 carriers, and it is thus also possible that some effects of apoE4 are mediated via loss of function.

Thus, there is a need in the art for compositions and methods that can counteract the gain of toxic effect of apoE4 in the treatment or prevention of neurodegenerative conditions. In particular, there is need in the art for immunotherapy methods and compositions directed against the apoE4 protein, which are specific and effective in the treatment or prevention of various neurodegenerative conditions, such as, Alzheimer's disease as well as such conditions that may be caused by closed or open head or brain injury or trauma.

### SUMMARY OF THE INVENTION

The present invention, provides methods and compositions for treating or preventing neurodegenerative condition by systemic administration of specific antibodies directed against apoE4 (anti-apoE4) to a subject in need thereof. In some embodiments, the neurodegenerative condition is caused by a disease, such as, Alzheimer's disease. In some embodiments, the neurodegenerative condition is induced by various head or brain traumatic injuries, such as, closed or open head injury. In some embodiments, the apoE4 antibody is a monoclonal antibody, specifically recognizing, interacting and/or binding the apoE4 protein. In some embodiments, a subject in need thereof is a subject that expresses the apoE4 gene.

According to some embodiments, the methods disclosed herein comprise the use of specific apoE4 antibody in a combination with at least one additional agent capable of modulating expression of apoE gene or protein. In some embodiments, the at least one additional agent may be capable of elevating the expression level of apoE proteins so as to increase the apoE variants other than apoE4. In some embodiments, the at least one additional agent may be capable of changing the extent of lipidation of apoE4. In some embodiments, the agent is Bexarotene.

In some embodiments, the methods disclosed herein comprise use of apoE4 monoclonal antibody in a combination with at least one additional agent capable of modulating expression of apoE gene or protein. In some embodiments, the at least one additional agent may be capable of elevating the expression level of apoE proteins.

According to some embodiments, the present invention is based, in part, on the unexpected and surprising finding that apoE4 antibodies administered systemically, as exemplified hereinbelow by intraperitoneal injection (i.p.), can counteract various brain related pathological phenotypes induced by apoE4, such as, hyperphosphorylation of tau.

Since the concentration of apoEs in the blood is high, it would have been expected that all the peripherally, i.e., systemically, injected apoE4 mAbs would have been bound to the blood apoE4 (i.e. be titrated) and thus prevent the antibodies from further reaching the CNS and/or affecting brain apoE. Thus, as further exemplified herein, it is surprising and unexpected that peripherally/systemically administered apoE4 antibodies can effect apoE4 in the brain, either directly (by reaching and entering the brain, i.e. crossing the blood-brain barrier (BBB)), or indirectly (by a mechanism which does not depend on the penetration of the mAbs into the brain). In further embodiments, the invention is based, in part, on the unexpected and surprising finding that systemic administration of specific antibodies directed against the apoE4 protein, which is localized to the brain, are able to exert an effect in the brain. In other embodiments, the invention is additionally based, in part, on the unexpected finding that systemic administration of monoclonal antibodies (apoE4) in animal models (e.g., by i.p. administration) results in the prevention and reversal of various pathological phenotypes induced by apoE4. Without wishing to be bound by any theory or mechanism of action, the activity of the apoE4 monoclonal antibody in treating or protecting against neurodegenerative conditions, such as, Alzheimer disease, may be associated with the activity of such antibody in blocking, preventing or weakening any of the specific effects of apoE4. Exemplary effects of apoE4 that may be affected by the specific antibody include, for example: 1) interfere with the ability of apoE4 to increase the neurodegenerative effects of Abeta (since apoE4 blocks the removal of Abeta (Aβ)) from the brain and activates its uptake into neurons following activation of the amyloid cascade; 2) apoE4 induces tau hyper phosphorylation which is known to be pathological; 3) ApoE4 renders the brain particular susceptible to lipid diets which contain low levels of ω3 polyunsaturated fatty acids; 4) apoE4 impairs mitochondrial and lysosomal functions. Reversal of all/part of these pathological effect of apoE4 can be achieved by systemic administration of specific antibodies against apoE4, and hence be used for the treatment or prevention of various neurological conditions.

According to some embodiments, there are thus provided methods for treating, ameliorating or preventing various neurodegenerative conditions. The neurodegenerative condition may be, for example, a neurodegenerative disease (such as, Alzheimer disease (AD)) and/or may be the result of a trauma to the head or brain (such as, traumatic brain injury (TBI), open head injury, closed head injury, and the like).

According to some exemplary embodiments, the present invention provides a method of treating or preventing Alzheimer Disease (AD), the method comprising systemically administering to a subject in need thereof an effective amount of a specific apoE4 antibody; thereby treating the Alzheimer disease.

In some embodiments, there is provided an antibody specific for apolipoprotein isoform apoE4 (apoE4 antibody) for use in the treatment or prevention of a neurodegenerative condition, by systemic administration of the antibody.

In other embodiments, the present invention further provides a method of treating or preventing a neurodegenerative condition caused or induced by a head or brain injury, the method comprising systemically administering to a subject in need thereof an effective amount of a specific apoE4 antibody; thereby treating the neurodegenerative condition. In some embodiments, administration of the specific apoE4 antibody may be performed at any time period after the head or brain injury 5

According to some embodiments, the apoE4 antibody is selected from a monoclonal antibody, a humanized antibody, a chimeric antibody, a single chain antibody, and antigen-binding fragments thereof. Each possibility is a separate embodiment. In some embodiments, the apoE4 antibody is a monoclonal antibody. In some embodiments, the monoclonal antibody may be selected from commercially available sources, e.g.: clones 9D11, 5G7, and the like.

According to some embodiments, the systemic administration is carried out by a route selected from the group consisting of: intraperitoneal, intravenous, subcutaneous, intranasal, and combinations thereof.

According to some embodiments the method further comprises administering at least one agent capable of modulating expression of proteins of the apoE family. In some exemplary embodiments, the at least one agent capable of modulating expression of apoE family is Bexarotene.

According to additional embodiments, there is further provided a method for preventing a neurodegenerative condition, the method comprising determining the expression of apolipoprotein isoform apoE4 in a subject; and systemically administering a subject expressing apoE4 an effective amount of an antibody specific for apoE4; thereby preventing the neurodegenerative condition.

According to some embodiments, there is provided an antibody specific for apolipoprotein isoform apoE4 for use in preventing a neurodegenerative condition by systemic administration; wherein the expression level of apoE4 in a subject is determined prior to the use of the antibody.

According to yet further embodiments, there is provided a method for screening and identifying a subject at risk of having a neurodegenerative condition, the method comprising detecting expression of apoE4 protein, and/or of apoE4 related molecular and structural changes in the retina of the subject, thereby identifying a subject at risk of having a neurodegenerative condition.

In yet further embodiments, the present invention further provides a method of treating or preventing a neurodegenerative condition caused or induced by a head or brain injury, the method comprising administering to a subject in need thereof an effective amount of a specific apoE4 antibody; thereby treating the neurodegenerative condition. The administration may be localized (for example, to the brain) or systemic (for example, by parenteral route).

### BRIEF DESCRIPTION OF THE DRAWINGS

FIGS. 1A-C: Effects of ApoE genotype on performance in the dry version of the Morris Water Maze of young ApoE3 and ApoE4 mice. Naïve ApoE3 and apoE4 mice, at 4 months of age, were subjected to the dry version of the Morris Water Maze. The mice were subjected to 4 daily trials and the latency to the water filled well was measured. The experiment was performed in 2 phases, such that after the 8^{th} day (arrow) the location of the water filled well was altered. The Figures show graphs representing the average of performance ApoE3 and ApoE4 mice (n=6) (latency time (seconds) vs. days. FIG. 1A represents the results as the daily average. FIG. 1B represents the results of the first trial of each day; FIG. 1C represents the results of the last (4^{th}) trial of each day. Quantification of the results is displayed (mean+/− SE). Empty squares correspond to ApoE3 mice whereas full squares correspond to ApoE4 mice. * indicate P<0.05;

FIGS. 2A-B: Effects of ApoE genotype on Vglut1 immuno-reactivity in the hippocampus of human ApoE targeted replacement mice. Naïve ApoE3 and ApoE4, mice, at 4 months of age, were sacrificed and their brains extracts subjected to western blot analysis. Western Blots of samples of whole hippocampi from ApoE3 and ApoE4 mice were immunostained with anti-Vglut antibody (n=11). FIG. 2A, shows pictogram of representative bands corresponding to Vglut1 and GAPDH. FIG. 2B shows bar graph quantification (mean+/− SE) of the results presented in FIG. 2A. * indicate P<0.05. All results are normalized to the 4 months old ApoE3 mice;

FIGS. 3A-C: Immunohistochemical assessment of the effects of ApoE genotype on the expression level and distribution of Vglut1 in the hippocampus of human ApoE targeted replacement mice. Naïve 4 months old ApoE3 and ApoE4 brain slices were immunostained with anti-Vglut 1 antibody. Representative coronal sections of 4 months old mice are presented, as well as their quantification. FIG. 3A shows results for the CA3 region; FIG. 3B shows results for the CA1 region and FIG. 3C shows results for the DG region. For each Figure, Representative coronal immunostained sections (with anti-Vglut1 antibody) of 4 months old mice are presented on the left panel, quantification of the immunostained sections of 4 months old mice is presented in the middle panel (mean+/− SE). Quantification of the immunostained sections of mice at 1, 2 and 4 months of age are presented on the right hand panel. White bars correspond to ApoE3 mice whereas black bars correspond to ApoE4 mice. Arrows indicate the area analyzed. * indicate P<0.05. All results are normalized to the 4 months old ApoE3 mice.

FIGS. 4A-C: Effects of ApoE genotype on Aβ42 immuno-reactivity in the hippocampus of human ApoE targeted replacement mice. Naïve 4 months old ApoE3 and ApoE4 brain sections were immunostained with anti-Aβ42 antibody. FIG. 4A shows results for the CA3 region; FIG. 4B shows results for the CA1 region; and FIG. 4C shows results for the DG region. For each Figure, Representative coronal immunostained sections (with anti-Aβ42 antibody) of 4 months old mice are presented on the left panel, quantification of the immunostained sections of 4 months old mice is presented in the middle panel (mean+/− SE). Quantification of the immunostained sections of mice at 1, 2 and 4 months of age are presented on the right hand panel. White bars correspond to ApoE3 mice whereas black bars correspond to ApoE4 mice. Arrows indicate the area analyzed. * indicate P<0.05. All results are normalized to the 4 months old ApoE3 mice.

FIGS. 5A-C: Effects of ApoE genotype on AT-8 immuno-reactivity in the hippocampus of human ApoE targeted replacement mice. Naïve 4 months old ApoE3 and ApoE4 brains were immunostained with the monoclonal antibody AT-8, which recognize phosphorylated tau (at positions-202/205). FIG. 5A shows results for the CA3 region; FIG. 5B shows results for the CA1 region; and FIG. 5C shows results for the DG region. For each Figure, Representative coronal immunostained sections (with anti-202/205 phospho Tau antibody) of 4 months old mice are presented on the left panel, quantification of the immunostained sections of 4 months old mice is presented in the middle panel (mean+/− SE). Quantification of the immunostained sections of mice at 1, 2 and 4 months of age are presented on the right hand panel. White bars correspond to ApoE3 mice whereas black bars correspond to ApoE4 mice. Arrows indicate the area analyzed. * indicate P<0.05. All results are normalized to the 4 months old ApoE3 mice.

FIG. 6A-E: Effects of ApoE genotype on VEGF immuno-reactivity in different brain regions of human ApoE targeted replacement mice. Naïve 4 months old ApoE3 and ApoE4 brain slices were immunostained with anti-VEGF. The results are presented in FIGS. 6A-E, which illustrates bar graphs quantification of immunostaining intensity by the anti-VEGF antibody in different regions: FIG. 6A—CA3; FIG. 6B—CA1; FIG. 6C—DG; FIG. 6D—Visual cortex; FIG. 6E—Entorhinal cortex (E). The quantification of 4 months old mice is presented (mean+/− SE). White bars correspond to ApoE3 mice whereas black bars correspond to ApoE4 mice. * indicate P<0.05. All results are normalized to the ApoE3 mice.

FIG. 7: The effects of apoE genotype on the basal gene expression levels of HIF transcription factors and VEGF ligands and receptors. mRNA extracted from the hippocampus of ApoE3 and apoE4 targeted replacement non-treated (Naïve) mice was used in RT-PCR analysis, with specific primers for Hif1, Hif2, VegfA, VegfB, FLT1 or kdr. Relative expression levels (arbitrary units) of the various tested genes in the hippocampus of ApoE3 or ApoE4 mice is represented in the bar graph shown in FIG. 7.; * p≦0.05, ** p=0.005

FIGS. 8A-B: Effects of ApoE genotype on TOM40 and COX1 in the CA3 of the hippocampus of human ApoE targeted replacement mice. Naïve ApoE3 and ApoE4, mice, at 4 months of age, were sacrificed and their brains extracts subjected to western blot analysis. Western Blots of samples of whole hippocampi from ApoE3 and ApoE4 mice were immunostained with anti-TOM40 or anti-COX1 antibodies. Results are presented for the CA3 subfield (region). FIG. 8A shows pictogram of representative immuno bands corresponding to Tom40 and Cox1, in the ApoE3 and ApoE4 mice. FIG. 8B shows bar graph quantification (mean+/− SE) of the results presented in FIG. 8A. White bars correspond to ApoE3 mice whereas black bars correspond to ApoE4 mice. * indicate P<0.05.

FIGS. 9A-B: Immunohistochemical assessment of the Effects of ApoE genotype on the levels TOM40 immuno-reactivity in the CA3 of the hippocampus of ApoE targeted replacement mice. Naïve 1, 2 and 4 months old ApoE3 and ApoE4 brain sections were immunostained with anti-TOM40 antibody. FIG. 9A shows pictograms of immunostaining of representative coronal sections of 1, 2 and 4 months old mice on the CA3 region. FIG. 9B show bar graphs of quantification of the FIG. 9A (mean+/− SE). White bars correspond to ApoE3 mice whereas black bars correspond to ApoE4 mice. * indicate P<0.05. All results are normalized to the 4 months old ApoE3 mice.

FIGS. 10A-C: Effects of ApoE genotype on ApoE receptors in the CA3 and CA1 of hippocampus of human ApoE targeted replacement mice. Naïve 4 months old ApoE3 and ApoE4 brains were immunostained with anti-LRP and anti-ApoEr2 antibodies. The results are presented in FIGS. 10A-C which illustrate bar graphs quantification (mean+/− SE) of immunostaining intensity by anti-LRP or anti-ApoEr2 antibodies: FIG. 10A—expression of ApoEr2 in the CA1 region; FIG. 10B—expression of ApoEr2 in CA3 region; FIG. 10C—expression of LRP in the CA1 region. White bars correspond to ApoE3 mice whereas black bars correspond to ApoE4 mice. * indicate P<0.05. All results are normalized to the 4 months old ApoE3 mice.

FIGS. 11A-D: Effects of ApoE genotype on mitochondrial and Lysosomal ultrastructure in hippocampus of young (3 months old) apoE4 mice. FIG. 11A: confocal microscopy images of cells showing mitochondrial ultrastructure in ApoE3 (top panel) or ApoE4 (lower panel) mice. FIG. 11B: confocal microscopy images of cells showing lysosomal ultrastructure in ApoE3 (top panel) or ApoE4 (lower panel) mice. FIG. 11C: Pictograms of immunohistochemical analysis of expression of Cathepsin D in hippocampus of apoE3 or apoE4 mice. FIG. 11D: pictograms showing immunoblots of expression of the autophagy substrate P62 in ApoE3 or ApoE4 mice.

FIGS. 12A-E: The effect of apoE genotype on synaptic parameters in the inner plexiform layer (IPL) of the retina. Cross section of apoE3 and apoE4 retinas were stained with Synaptophysin (total synapses), Vglut (marker for excitatory synapses) and Vgat (marker for inhibitory synapses) antibodies. The results are shown in the pictograms of FIG. 12A. IPL is marked with white arrow bars. FIGS. 12B-E shows bar graphs of quantitative analysis of the various synaptic markers/parameters: Synaptophysin (FIG. 12B); Vglut (FIG. 12C); Vgat (FIG. 12D); Vglut/Vgat ratio (FIG. 12E). *p<0.03. White bars correspond to ApoE3 mice whereas black bars correspond to ApoE4 mice.

FIGS. 13A-E: The effect of apoE genotype on Neuronal parameters in the retina. Cross section of apoE3 and apoE4 retinas were stained for bipolar cells (with anti-αCHX10 antibody), Rod bipolar cells (with anti-αPKCα), amacrine and Ganglion cells (GCL) with PAX6 as a marker. The results are presented in the pictograms shown in FIG. 13A. Left hand arrows in right hand panel are indicative of amacrine and right hand arrows in the right hand panel are indicative of GCL. Further shown in FIGS. 13B-E are bar graphs of quantitative analysis of the different neuronal markers/parameters: FIG. 13B—Bipolar cells; FIG. 13C—Rod bipolar cells; FIG. 13D—Amacrine; FIG. 13E—GCL. *p<0.03, **P<0.01. White bars correspond to ApoE3 mice whereas black bars correspond to ApoE4 mice.

FIGS. 14A-B: Effects of intracerebroventricular (i.c.v.) administration of apoE4 monoclonal antibody on the accumulation of Aβ and the associated increase in the levels of the apoE receptor, LRP, in hippocampal neurons of transgenic mice. Naïve apoE3, and apoE4 mice, were administered by i.c.v. injection with: ACSF injection (sham), injection with thiorphan (neprilyin inhibitor, Treated), injection of thiorphan together with non-specific monoclonal antibody (NS), or apoE4 monoclonal antibody (9D11) for one week utilizing Alzet miniosmotic pumps. Results are presented in FIGS. 14A-B, which show bar graphs of quantification of expression levels (arbitrary units) of Aβ42 (FIG. 14A)) or LRP1 (FIG. 14B), in CA1 neurons. Results are shown as mean+/− SE. White bars correspond to ApoE3 mice, and black bars correspond to ApoE4 mice.

FIGS. 15A-E: Effect of intraperitoneal (i.p.) injection of apoE4 monoclonal antibody on phosphorylated tau in the hippocampus of transgenic mice. Naïve apoE3, and apoE4 mice, were weekly administered by i.p. injection with: PBS injection, injection of non-specific monoclonal antibody (mAb), or apoE4 monoclonal antibody (9D11) for weeks up to 4 months of age. Brain slices of the mice were subjected to immunohistochemical analysis using AT-8 antibody (n=4-6). Results are presented in FIGS. 15A-E, which show representative AT8 stained coronal sections of the CA3 or DG subfield, respectively, of apoE3 and apoE4 mice treated with non specific IgG and of apoE4 mice treated with apoE4 mAb (FIGS. 15A and 15C, respectively), and bar graphs of quantification of expression of AT-8 in CA3 region (FIG. 15B), DG (FIG. 15D) and CA1 (FIG. 15E). The results are displayed as mean+/− SE of expression of AT-8 at various brain regions of E3 or E4 mice, under the indicated treatments. White bars correspond to ApoE3 mice, and black bars correspond to ApoE4 mice. * indicate P<0.05 between ApoE3 and ApoE4, # indicate p<0.05 between ApoE4 mice injected with non-specific mAb and the specific anti-apoE3 monoclonal antibody (9D11). The results are normalized to the PBS injected ApoE3 mice.

FIGS. 16A-I: Effect of intraperitoneal (i.p.) injection of apoE4 monoclonal antibody on Aβ42 or Vglut1 in the hippocampus of transgenic mice. Naïve ApoE3, and apoE4 mice, underwent weekly intraperitoneal (i.p.) injections of PBS, non-specific monoclonal antibody (mAb), or apoE4 monoclonal antibody (9D11) for weeks up to 4 months of age. Brain slices of the mice were subjected to immunohistochemical analysis using anti-Aβ42 or anti-Vglut1 antibodies (n=4-6). Results are presented in FIGS. 16A-E, which show representative pictograms of Aβ42 stained (mAb G2-11) coronal sections of the CA3 (FIG. 16A) or DG (FIG. 16C) subfields of apoE3 and apoE4 mice treated with non specific IgG and of apoE4 mice treated with the apoE4 mAb and of bar graphs of quantification of expression of Aβ42 in CA3 (FIG. 16B), DG (FIG. 16D), and CA1 (FIG. 16E). FIGS. 16F-I, show representative pictograms of VGluT1 stained coronal sections of the CA3 (FIG. 16F) or DG (FIG. 16H) subfields of apoE3 and apoE4 mice treated with non specific IgG and of apoE4 mice treated with the apoE4 mAb and of bar graphs of quantification of expression of VGluT1 in CA3 (FIG. 16G), DG (FIGS. 16H & 16I), and CA1 (FIG. 16J). The results are displayed as mean+/− SE of expression of Aβ42 or Vglut1 at the various brain regions of E3 or E4 mice, under the indicated treatments. White bars correspond to ApoE3 mice, and black bars correspond to ApoE4 mice. * indicate P<0.05 between ApoE3 and ApoE4 mice. All results are normalized to the PBS injected ApoE3 mice.

FIGS. 17A-B: Effect of intraperitoneal (i.p.) injection of apoE4 monoclonal antibody on apoER2 receptor in TR mice. Naïve ApoE3, and apoE4 mice, underwent weekly intraperitoneal (i.p.) injections of PBS, non-specific monoclonal antibody (mAb; mg/kg), or apoE4 monoclonal antibody (9D11; 10 mg/kg) for weeks up to 4 months of age. Brain slices of the mice were subjected to immunohistochemical analysis using anti-ApoER2 (n=4-6). The analysis was performed in the CA3 hippocampal subregion of 4 months old apoE3 or apoE4 mice. Results are presented in FIGS. 17A-B, which show representative pictograms of ApoER2 stained coronal sections of the CA3 subfield of apoE3 and apoE4 mice treated with non specific IgG and of apoE4 mice treated with the apoE4 mAb (FIG. 17A). The results shown in FIG. 17B show bar graph of quantification of expression of ApoER2 receptor in CA3. White bars correspond to ApoE3 mice, and black bars correspond to ApoE4 mice. # indicate P<0.05 between ApoE3 and ApoE4 mice. All results are normalized to the PBS injected ApoE3 mice.

FIG. 18A-B: effect of intraperitoneal (i.p.) injection of apoE4 monoclonal antibody on cognitive ability of TR mice. Naïve ApoE3, and apoE4 mice, underwent weekly intraperitoneal (i.p.) injections of PBS, non-specific monoclonal antibody (mAb), or apoE4 monoclonal antibody (9D11) for weeks up to 4 months of age. The mice were then tested in the novel object recognition test and the Morris water maze test. Results of the novel object recognition test are presented in FIG. 18A, showing the time near novel object (%) of apoE3 (white bars) or apoE4 (black bars), treated with non-specific antibody or with apoE4 monoclonal antibody. # indicates p<0.05 of a Student's T-test comparison between treatments of apoE4, and ** indicates p<0.01 of a Student's T-test comparison between apoE3 and apoE4. Results of the Morris maze test are presented in FIG. 18B, showing the latency to platform time (seconds) on Days 1-3, of apoE3 or ApoE4 mice, treated with non-specific antibody (NS, black bars) or with apoE4 monoclonal antibody (9D, white bars). *p<0.05 for a T-test comparison between treatments).

FIGS. 19A-I: Effects of intraperitoneal (i.p.) injection of apoE4 monoclonal antibody on the levels of IgG and of ApoE in the hippocampus of apoE4 and apoE3 targeted replacement mice. Naïve ApoE3 and apoE4 mice underwent weekly intraperitoneal (i.p.) injections of: PBS (“PBS”), non-specific monoclonal antibody (“NS”), or apoE4 monoclonal antibody (“aE4”, 9D11) for weeks up to 4 months of age. Brain slices of the mice were subjected to immunohistochemical analysis using IgG and hApoE antibodies (n=4-6). Results are presented in FIGS. 19A-C, which show bar graphs of quantification of expression of IgG in CA3 (FIG. 16A), DG (FIG. 19B), and Hilus of DG (FIG. 19C). FIGS. 19D-F, show bar graphs of quantification of expression of hApoE in CA3 (FIG. 19D), DG (FIG. 19E), and Hilus of DG (FIG. 19F). FIGS. 19G-I, show bar graphs of quantification of co-localization of IgG and hApoE in CA3 (FIG. 19G), DG (FIG. 19H), and Hilus of DG (FIG. 19I). The results are displayed as mean+/− SE. White bars correspond to ApoE3 mice, and black bars correspond to ApoE4 mice. * indicate P≦0.01. All results are normalized to the PBS injected ApoE3 mice.

FIGS. 20A-D: Treatment effect of intraperitoneal (i.p.) injection of apoE4 monoclonal antibody. Three and a half months old apoE3 and apoE4 homozygote mice were i.p. injected with: PBS, non-specific (NS) IgG (MOPC-21, Bio X-Cell), or the apoE4 9D11mAb (10 mg/kg). The mice were i.p. injected 3 times per week, for two weeks. The mice were sacrificed at the age of four months. Brain slices of the mice were subjected to biochemical and immunohistochemical analysis. The results presented in FIGS. 20A-D show bar graphs of quantification of relative expression in the CA3 hippocampal subregion of the mice of the following markers: phosphorylated tau (AT-8, FIG. 20A), Aβ42 (Amyloid β1-42, FIG. 20B), VGluT-1 (vasicular glutamate transporter −1, FIG. 20C), and ApoE receptor 2 (apoER2, FIG. 20D). The results are displayed as mean+/− SE of relative expression compared to the corresponding expression in apoE3 mice, treated with non-specific antibody (NS). White bars correspond to ApoE3 mice, and black bars correspond to ApoE4 mice. NS—treatment with non-specific antibody, 9D—treatment with specific apoE4 antibody (D911). # indicates p<0.05 at a Student's T-test comparison between treatments of ApoE4.

FIGS. 21A-C: Effect of Bexarotene on expression levels of various apoE related genes in apoE3 and apoE4 mice. ApoE3, and apoE4 mice were orally gavaged with either water or Bexarotene (100 mg per kg per day, (Eisai Inc. (Targretin)) for days. After days, the mice were sacrificed and brains were excised, and further processed by immunoblot and/or immunohistochemistry. The results presented in FIGS. 21A-C show the expression levels of apoE (FIG. 21A) apoE-lapidating gene, ABCG1 (FIG. 21B) and apoE-lapidating gene ABCG1 (FIG. 21C). The results shown in FIGS. 21A-C, are of representative Western blots images (top panels, respectively) and bar graphs of quantification of the corresponding blots (lower panels) of the expression of ApoE (FIG. 21A), ABCG1 (FIG. 21B) and ABCA1 (FIG. 21C), in samples obtained from control mice (Control) or Bexarotene treated mice (Bexarotene) (n=5). The results are displayed as mean+/− SE. White bars correspond to ApoE3 mice, and black bars correspond to ApoE4 mice. *p<0.05 for differences between apoE3 and apoE4 and #p<0.05 for effects of Bexarotene.

FIGS. 22A-D: Effect of Bexarotene on accumulation of several markers which have been shown to be affected by apoE4. Three and a half months old ApoE3 and apoE4 mice were orally gavaged with either water or Bexarotene (100 mg per kg per day, (Eisai Inc. (Targretin)) for days. After days, the mice were sacrificed and brains were excised, and further processed by immunoblot and/or immunohistochemistry. FIG. 22A shows the effect of Bexarotene on the accumulation of Aβ42. The results presented in on the left hand panel of FIG. 22A show expression of Aβ42 (as determined by immunostaining with anti-Aβ42 antibody) in representative brain sections of untreated apoE3 (top) or ApoE4 (bottom) mice. The bar graphs on the right hand panel of FIG. 22A show quantization of the results (arbitrary units, a.u.), and the difference between the control (water-treated mice) and mice treated with Bexarotene (n=6). The results are displayed as mean+/− SE. White bars correspond to ApoE3 mice, and black bars correspond to ApoE4 mice. *p<0.05 for differences between apoE3 and apoE4 and #p<0.05 for effects of Bexarotene. FIG. 22B shows the effect of Bexarotene on tau hyperphosphorylation. The results presented on the left hand panel of FIG. 22B show phoshprylated tau (identified by the AT-8 antibody staining), in representative brain sections of untreated apoE3 (top) or ApoE4 (bottom) mice. The bar graphs on the right hand panel of FIG. 22B show quantization of the results (arbitrary units, a.u.), and the difference between the control (water-treated mice) and mice treated with Bexarotene (n=6). The results are displayed as mean+/− SE. White bars correspond to ApoE3 mice, and black bars correspond to ApoE4 mice. *p<0.05 for differences between apoE3 and apoE4 and #p<0.05 for effects of Bexarotene. FIG. 22C shows the effect of Bexarotene on the expression levels of the presynaptic transporter VGlut. The results presented in on the left hand panel of FIG. 22C show VGlut expression (as determined by immunostaining) in representative brain sections of untreated ApoE3 or ApoE4 mice. The bar graphs on the right hand panel of FIG. 22C show quantization of the results in arbitrary units (a.u.), and the difference between the control (water-treated mice) and mice treated with Bexarotene (n=6). The results are displayed as mean+/− SE. White bars correspond to ApoE3 mice, and black bars correspond to ApoE4 mice. *p<0.05 for differences between apoE3 and apoE4 and #p<0.05 for effects of Bexarotene. FIG. 22D shows the effect of Bexarotene on the levels of apoE receptor, apoER2. The results presented on the left hand panel of FIG. 22D show apoER2 expression in representative brain sections of untreated apoE3 or ApoE4 mice. The bar graphs on the right hand panel of FIG. 22D show quantization of the results in arbitrary units (a.u.), and the difference between the control (water-treated mice) and mice treated with Bexarotene (n=6). The results are displayed as mean+/− SE. White bars correspond to ApoE3 mice, and black bars correspond to ApoE4 mice. *p<0.05 for differences between apoE3 and apoE4 and #p<0.05 for effects of Bexarotene.

FIGS. 23A-D: Effect of Bexarotene on apoE4 induced behavioral deficits. ApoE3 and ApoE4 mice were orally gavaged with either water or Bexarotene (100 mg per kg per day, (Eisai Inc. (Targretin)) for days. After days, the mice were subjected to behavioral tests. FIG. 23A shows the effect of Bexarotene on object recognition impairment in apoE4 mice, as measured by the object recognition test. The bar graphs presented in FIG. 23A show the percent of time spent near the new object (%) of control apoE3 or apoE4 mice (control) or Bexarotene treated apoE3 or apoE4 mice (Bexarotene). The results are displayed as mean+/− SE. White bars correspond to ApoE3 mice, and black bars correspond to ApoE4 mice. *p<0.05 for differences between apoE3 and apoE4 and #p<0.05 for effects of Bexarotene. FIGS. 23B-C show the effect of Bexarotene on apoE4 induced behavioral deficits, as measured by the Morris swim test. The Morris swim test monitors the extent to which the mice learn the position of a hidden platform in a water filled arena. The line graphs presented in FIG. 23B, show the time (Seconds) it took the tested mice to reach the platform of: control (water-treated) apoE3 mice (E3W), control (water-treated apoE4 mice (E4W), Bexarotene treated apoE3 mice (E3B) and Bexarotene treated apoE4 mice (E4B). The test was repeated daily over the course of four days (days 1-4). The results are displayed as mean+/− SE. *p<0.05 for differences between groups. The bar graphs presented in FIG. 23C, demonstrate the time (seconds) the tested mice spent on looking/searching for the missing platform (that has been removed), in its previous location. The tested mice groups were: control apoE3 (treated with water), control apoE4 mice (treated with water), Bexarotene treated apoE3 mice and Bexarotene treated apoE4 mice. The results are displayed as mean+/− SE. White bars correspond to ApoE3 mice, and black bars correspond to ApoE4 mice. *p<0.05 for differences between groups. FIG. 23D shows the effect of Bexarotene on stress induced by a mild electric shock in apoE4 mice (fear conditioning test). In these experiments the mice are given a small shock in day 1, and then re-introduced to the same environment (arena). The extent to which the mice freeze when re-entered to the test arena is considered a measure of their memory. The bar graphs presented in FIG. 23D, show the time (seconds) the tested mice froze upon re-entry to the test arena. The tested mice groups were: control apoE3 (treated with water), control apoE4 mice (treated with Water), Bexarotene treated apoE3 mice and Bexarotene treated apoE4 mice. The results are displayed as mean+/− SE. White bars correspond to ApoE3 mice, and black bars correspond to ApoE4 mice. *p<0.05 for differences between groups (n=7).

### DETAILED DESCRIPTION OF THE INVENTION

### Definitions

The term “antibody” (also referred to as an “immunoglobulin”) is used in the broadest sense and specifically encompasses monoclonal antibodies (including full length monoclonal antibodies) and antibody fragments so long as they exhibit the desired biological activity. “Antibody fragments” comprise a portion of a full length antibody, generally the antigen binding or variable region thereof. Examples of antibody fragments include Fab, Fab′, F(ab′)2, and Fv fragments; diabodies; linear antibodies; single-chain antibody molecules; and multispecific antibodies formed from antibody fragments.

The basic unit of the naturally occurring antibody structure is a heterotetrameric glycoprotein complex of about 150,000 daltons, composed of two identical light (L) chains and two identical heavy (H) chains, linked together by both noncovalent associations and by disulfide bonds. Each heavy and light chain also has regularly spaced intrachain disulfide bridges. Five human antibody classes (IgG, IgA, IgM, IgD and IgE) exist, and within these classes, various subclasses, are recognized on the basis of structural differences, such as the number of immunoglobulin units in a single antibody molecule, the disulfide bridge structure of the individual units, and differences in chain length and sequence. The class and subclass of an antibody is its isotype.

The amino terminal regions of the heavy and light chains are more diverse in sequence than the carboxy terminal regions, and hence are termed the variable domains. This part of the antibody structure confers the antigen-binding specificity of the antibody. A heavy variable (VH) domain and a light variable (VL) domain together form a single antigen-binding site, thus, the basic immunoglobulin unit has two antigen-binding sites. Particular amino acid residues are believed to form an interface between the light and heavy chain variable domains (Chothia et al., J. Mol. Biol. 186, 651-63 (1985); Novotny and Haber, (1985) Proc. Natl. Acad. Sci. USA 82 4592-4596).

The carboxy terminal portion of the heavy and light chains form the constant domains i.e. CH1, CH2, CH3, CL. While there is much less diversity in these domains, there are differences from one animal species to another, and further, within the same individual there are several different isotypes of antibody, each having a different function.

In some embodiments, an antibody is a monoclonal, recombinant or modified antibody, e.g., a chimeric, humanized, deimmunized or an in vitro generated antibody. The term “recombinant” or “modified” antibody as used herein is intended to include all antibodies that are prepared, expressed, created, or isolated by recombinant means, such as (i) antibodies expressed using a recombinant expression vector transfected into a host cell; (ii) antibodies isolated from a recombinant, combinatorial antibody library; (iii) antibodies isolated from an animal (e.g. a mouse) that is transgenic for human immunoglobulin genes; or (iv) antibodies prepared, expressed, created, or isolated by any other means that involves splicing of human immunoglobulin gene sequences to other DNA sequences. Such recombinant antibodies include humanized, CDR grafted, chimeric, deimmunized, and in vitro generated antibodies; and can optionally include constant regions derived from human germline immunoglobulin sequences.

In some embodiments, an antibody does not comprise a full-length immunoglobulin heavy chain and a full-length immunoglobulin light chain, and instead comprises antigen-binding fragments of a full-length immunoglobulin heavy chain and a full-length immunoglobulin light chain. In some embodiments, the antigen-binding fragments are contained on separate polypeptide chains; in other embodiments, the antigen-binding fragments are contained within a single polypeptide chain. The term “antigen-binding fragment” refers to one or more fragments of a full-length antibody that are capable of specifically binding to an apoE4 epitope. Examples of binding fragments include (i) a Fab fragment (a monovalent fragment consisting of the VL, VH, CL and CH1 domains; (ii) a F(ab′)₂ fragment (a bivalent fragment comprising two Fab fragments linked by a disulfide bridge at the hinge region; (iii) a Fd fragment (consisting of the VH and CH1 domains); (iv) a Fv fragment (consisting of the VH and VL domains of a single arm of an antibody); (v) a dAb fragment (consisting of the VH domain); (vi) an isolated CDR; (vii) a single chain Fv (scFv) (consisting of the VH and VL domains of a single arm of an antibody joined by a synthetic linker using recombinant means such that the VH and VL domains pair to form a monovalent molecule); (viii) diabodies (consisting of two scFvs in which the VH and VL domains are joined such that they do not pair to form a monovalent molecule; the VH of each one of the scFv pairs with the VL domain of the other scFv to form a bivalent molecule); (ix) bi-specific antibodies (consisting of at least two antigen binding regions, each region binding a different epitope). In some embodiments, a subject antibody fragment is a Fab fragment. In some embodiments, a subject antibody fragment is a single-chain antibody (scFv).

As referred to herein, the terms “ApoE”, “apoE” and “APOE” may interchangeably be used and are directed to the gene or gene product (i.e. protein) of apolipoprotein E.

As referred to herein, the terms “ApoE2”, “apoE2” and “APOE2” may interchangeably be used and are directed to the gene or gene product (i.e. protein) of the apolipoprotein E2 iso form.

As referred to herein, the terms “ApoE3”, “apoE3” and “APOE3” may interchangeably be used and are directed to the gene or gene product (i.e. protein) of the apolipoprotein E3 isoform.

As referred to herein, the terms “ApoE4”, “apoE4”, “apoe4” and “APOE4” may interchangeably be used and are directed to the gene or gene product (i.e. protein) of the apolipoprotein E4 isoform.

As referred to herein, the terms “anti-ApoE4”, “anti-apoE4”, “apoE4 antibody”, “anti apoE4 antibody”, “anti apoE4 Ab”, “an antibody specific for apolipoprotein isoform apoE4” and “anti-APOE4” may interchangeably be used and are directed to a specific antibody directed against the apoE4 protein and which is able to specifically recognize/bind/interact with the apoE4 protein. In some embodiments, the anti-apoE4 is a monoclonal antibody (mAb). In some embodiments, the binding affinity of anti-apoE4 to apoE4 is much higher than the binding to apoE3. In some embodiments, an apoE4 antibody does not recognize other isoforms of ApoE, such as ApoE3. In some embodiments, the apoE4 antibody is not bound or linked to a moiety which permits the antibody to traverse the Blood Brain Barrier (BBB). In some embodiments, the apoE4 antibody is not fused to a polypeptide that binds to an endogenous blood brain barrier (BBB) receptor.

ApoE4 antibodies are disclosed, for example, in U.S. Patent Application Publication No. 2013/0017251. Monoclonal anti-ApoE4 antibodies are disclosed, for example, in the PhD thesis of Gal Ophir, entitled “Mechanism underlying the pathological effect of apolipoproteinE4: the role of brain inflammation”, Tel Aviv University, Israel 2006. Monoclonal antibodies are available commercially for example, in Covance Data sheet Cat. no. SIG 39754 or Covance Data Sheet Cat. No. SIG 39752 (www.covance.com).

As referred to herein, the terms “apoE mice (mouse)” or “human ApoE targeted replacement mice” is directed to a transgenic, targeted replacement (TR) mice, which is created by gene targeting, whereby the human apoE gene was used to replace the mouse endogenous apoE gene. Thus, the terms “apoE3 mice” or “E3 mice/mouse” refer to transgenic mice in which the endogenous apoE gene is replaced by human apoE3. The terms “apoE4 mice” or “E4 mice” refer to transgenic mice in which the endogenous apoE gene is replaced by human apoE4. The apoE3 or apoE4 mice are homozygotes. A heterozygote harboring the human apoE3 and the apoE4 gene is referred to herein as E3/E4 mice or apoE3/apoE4 mice. The human apoE4 coding sequence has Accession No. AAB59397. Human apolipoprotein E (epsilon-4 allele) gene, complete cds has accession number M10065.

In some embodiments, the term “apoE engineered mice” is directed to include any type of genetically engineered mice that is modified to express an apoE4 gene (or a portion of the gene encoding for apoE4). For example, non targeted replacement mice, which express the human apoE4 gene on the background of an apoE Knockout mouse, and under the regulation of a variety of promoters such as NSE (neuronal expression), GFAP (astrocytes), or under the regulation of human regulatory elements.

The terms “treating” or “treatment” refer to administering a composition, which includes at least one reagent/substance, effective to ameliorate symptoms associated with a condition, to lessen the severity or cure the condition. In some embodiments, treatment is the reversal of the condition or any of ifs detectable symptoms or effects. In some embodiments, the effect of the treatment may be determined at a cellular level, at an organ level and/or at the organism (body) level.

The term “preventing” as used herein refers to avert or avoid a condition from occurring. In some embodiments, preventing is directed to ameliorating the damage associated with a condition, such as a neurodegenerative condition.

As referred to herein, the term “modulate” is directed to affecting (changing) the expression and/or activity of a nucleic acid and/or a polypeptide. The term modulating may refer to increasing and/or attenuating (down regulating) the expression and/or activity of the nucleic acid and/or the polypeptide.

The term “mammal” is directed to include any mammal, including, for example, pet animals, such as dogs and cats; farm animals, such as pigs, cattle, sheep, and goats; laboratory animals, such as mice and rats; primates, such as monkeys, apes, and chimpanzees; and humans.

The term “effective amount” with respect to the antibodies and/or active agent(s) of the invention should be understood as meaning an amount of each of these active agents required to achieve a therapeutic effect, without causing excessive or uncontrollable adverse side effects. The effective amount required to achieve the therapeutic end result may depend on a number of factors including, for example, the specific type of the neurodegenerative condition and the severity of the patient's condition. The effective amount (dose) of the active agent(s), in the context of the present invention should be sufficient to affect a beneficial therapeutic response in the subject over time.

The term “biological sample” as used in various embodiments and examples can be any appropriate body-derived sample. The sample may include fluid samples such as whole blood, peripheral blood monocytes, leukocytes. The samples may include various cells and tissues. The sample may include fixed and/or embedded tissue sections. The samples may be either freshly extracted or frozen. The samples may be obtained from living or dead subjects and may be obtained from any organism, such as, for example, humans, mice and rats.

The terms “systemic administration” and “systemic route” are used interchangeably and refer to any route of administration, other than directly into the Brain or CNS. In some embodiments, the term systemic administration excludes specifically intracerebroventricular (i.c.v.) administration route.

Combination therapy as used herein and in the claims may refer to any of a number of different combination treatments, including for example, substantially overlapping periods of administration of two or more treatments; simultaneous, sequential or successive administration of two or more treatments, scheduled administration of two or more treatments during alternating time periods, and the like.

According to some embodiments, a neurodegenerative condition may be selected from, but not limited to: Amyotrophic Lateral Sclerosis (ALS), Alzheimer's disease (AD), Primary Lateral Sclerosis (PLS), Spinal Muscular Atrophy (SMA), or any combination thereof. Each possibility is a separate embodiment.

In some embodiments, a neurodegenerative condition may include a condition caused by head or brain injury, such as, closed brain injury, open head injury, traumatic brain injury, and the like. Each possibility is a separate embodiment.

It has previously been shown that a synergistic interaction between apoE4 and the amyloid cascade are related to a gain of function, since the deposition and accumulation of Aβ is elevated in the apoE4 but not in apoE3 mice or apoE Knock-out (KO) mice. These effects can be inhibited by apoE4 Antibodies, when administered locally to the brain, for example, by i.c.v injection. However, as detailed above, in view of the high level of apoE4 in the circulating blood, it is highly unlikely that systemic administration of a specific apoE4 would exert an effect on the brain. Thus, the present invention is based in part on the unexpected and surprising finding that systemic administration of specific monoclonal apoE4 antibodies are able to reach and enter the brain, and furthermore, exert a detectable effect in the brain. In some embodiments, the detectable effect by the apoE4, may be the reversal of various phenotypes/conditions induced by the presence/expression of apoE4. Exemplary such phenotypes include, but are not limited to: Accumulation, oligomerization, and deposition of Aβ; tau phosphorylation; intraneuronal, lysosomal, and mitochondrial pathology, lysosomal, an autophagy impairments and synaptic and neuronal loss; Behavioral dysfunction, such as memory impairments; and the like.

According to some embodiments, there are thus provided methods for treating or preventing a neurodegenerative condition(s), by systemic administration of antibodies directed against apoE4 (apoE4), to a subject in need thereof. In some embodiments, the apoE4 antibody is a monoclonal antibody.

According to further embodiments, the systemic administration is carried out by a route selected from the group consisting of: intraperitoneal, intravenous, subcutaneous, intranasal, and combinations thereof.

Since the concentration of apoEs in the blood is high, it would have been expected that all the peripherally/systemically injected apoE4 mAbs would have been titrated by the blood apoE4 which would thus prevent the antibodies from further reaching the CNS and/or affecting brain apoE. Without wishing to be bound to any theory or mechanism, in mice, for example, serum volume is generally considered to have a volume of about 2 ml. Concentration of IgG in the serum is about 2 mg/ml, which corresponds to about 13 Micromolar. Hence, for example, for IgG (i.e., an antibody) administered systemically to the mice (for example, by intraperitoneal injection at an injection regime of 0.25 mg/weekly), even if assume that all of the injected IgG (i.e., the antibody) enters the blood stream, this equals to about 0.125 mg/ml. This is about 6% of the level of the total IgG in the blood and translates to about 0.8 Micromolar. As known in the art, the concentration of ApoE in the serum is about 100 microgram/ml, which equals about 4 micromolar, about half of which is apoE4. Accordingly, the concentration of apoE4 in the serum is several fold higher than the maximal estimate of the level of the injected IgG which could reach the serum. This means that there is large excess of apoE4 in the serum relative to the amount of injected apoE4 antibody, leaving no free (unbound) antibody available which can affect the apoE4 in the brains (for example, by penetrating the brain and directly interacting with the apoE4).

According to some embodiments the method further comprises administering at least one agent capable of modulating expression and/or lipidation of apoE proteins expression. In some exemplary embodiments, the at least one agent capable of modulating expression and/or lipidation of apoE expression is Bexarotene. Each possibility is a separate embodiment.

In some embodiments, a method which comprises a combination therapy of administering an apoE4 antibody in combination with an agent capable of modulating expression and/or lipidation of apoE proteins is beneficial, since, whereas the antibodies counteracts the gain of toxic effects induced by apoE4, the effect of the agent capable of modulating expression and/or the lipidation of apoE proteins (such as Bexarotene) is based on the overall beneficial effects of raising the expression levels and/or the lipidation of apoE proteins in general, of which some isoforms (such as, apoE3) may have beneficial effect.

In some embodiments, the neurodegenerative condition is a disease, such as, Alzheimer's disease.

In some embodiments, the neurodegenerative condition is a condition caused or induced by brain or head injury, such as, closed brain injury or open head injury. In some embodiments, the condition is caused or induced by traumatic brain injury (TBI).

In some embodiments, the present invention provides a method of treating or preventing a neurodegenerative condition caused or induced by a head or brain injury, the method comprising administering to a subject in need thereof an effective amount of a specific apoE4 antibody; thereby treating the neurodegenerative condition. In some embodiments, the administration is systemic (for example, by i.p.). In some embodiments, the administration is localized to the brain (for example, by i.c.v.).

In some embodiments, administration of the specific apoE4 antibody may be performed at any time period after the head or brain injury. For example, the administration of the specific apoE4 antibody may be performed within 1-60 minutes after the injury. For example, the administration of the specific apoE4 antibody may be performed within 0.5-48 hours after the injury. For example, the administration of the specific apoE4 antibody may be performed 1-7 days after the injury. For example, the administration of the specific apoE4 antibody may be performed 1-56 weeks after the injury. For example, the administration of the specific apoE4 antibody may be performed within one year or more after the injury.

According to yet further embodiments, a subject in need of a treatment is a subject carrying the apoE4 gene. In some embodiments, a subject in need of a treatment is a subject expressing the apoE4 gene in any tissue of his body. In some embodiments, the tissue is a neuronal tissue, such as, various brain regions, ocular tissue, retina, and the like. In some embodiments, a subject in need of a treatment is a subject expressing high levels of apoE4 protein in a tissue, such as, neuronal tissue, (for example, brain, ocular, retina), and the like. In some embodiments, a subject in need thereof is a subject expressing apoE4 in the brain (for example, in glia cells). According to some embodiments, a subject in need thereof is an infant, a new born or a child carrying the apoE4 gene (allel(s)) or expressing apoE4 protein in one or more tissues.

In other embodiments, a subject in need of a treatment is a subject having a detectable phenotype associated with expression of apoE4, such as, for example, but not limited to: accumulation, oligomerization, and deposition of Aβ in the brain; intraneuronal, lysosomal, and/or mitochondrial pathology in various regions of the brain, synaptic and neuronal loss in brain or other neuronal tissues.

According to some embodiments, there is thus provided a method for preventing a neurodegenerative condition, the method comprising determining the expression level of apoE4 in a biological sample of a subject; and based on the determination of the expression level of the apoE4, administering the subject with an effective amount of an apoE4 antibody; thereby preventing the neurodegenerative condition.

In some embodiments, the biological sample may be any tissue of the subject. In some embodiments, the biological sample is selected from a tissue or a bodily fluid In some embodiments, the tissue is a brain tissue. In some embodiments, the tissue is a retinal tissue. In some embodiments, the tissue is a neuronal tissue. In some embodiments, the tissue is a glia tissue. In some embodiments, the biological sample is blood sample.

In some embodiments, determining the expression level of apoE4 may be performed in any method known in the art that is used to determine expression level of a protein or a gene or gene transcript. Exemplary methods may include such methods as, but not limited to: Western Blot, immunohistochemistry, immunocytochemistry, Reverse transcription (RT)-PCR, and the like.

According to yet further embodiments, there is provided a method for screening and identifying a subject at risk of having a neurodegenerative condition, the method comprising detecting expression of apoE4 protein in the retina of the subject, thereby identifying a subject at risk of having a neurodegenerative condition.

In some embodiments, there is provided a method for identifying or screening a subject at risk of having a neurodegenerative condition, the method comprising determining the pathological status of the retina of the subject; and based on the pathological status, administer the patient with an effective amount of an apoE4 antibody.

It should be noted that according to the teaching of the present invention, the specific antibodies used in the methods of the present invention may be administered before, during, or after the administration of the at least one additional agent.

For use in the methods of the invention, the specific antibodies may be formulated in a conventional manner using one or more pharmaceutically acceptable carriers, stabilizers or excipients (vehicles) to form a pharmaceutical composition as is known in the art, in particular with respect to protein active agents. Carrier(s) are “acceptable” in the sense of being compatible with the other ingredients of the composition and not deleterious to the recipient thereof. Suitable carriers typically include physiological saline or ethanol polyols such as glycerol or propylene glycol.

The antibody may be formulated as neutral or salt forms. Pharmaceutically acceptable salts include the acid addition salts (formed with free amino groups) and which are formed with inorganic acids such as hydrochloric or phosphoric acids, or such organic acids such as acetic, oxalic, tartaric and maleic. Salts formed with the free carboxyl groups may also be derived from inorganic bases such as sodium, potassium, ammonium, calcium, or ferric hydroxides, and organic bases as isopropylamine, trimethylamine, 2-ethylamino ethanol, histidine and procaine.

The compositions may be suitably formulated for intravenous, intramuscular, subcutaneous, or intraperitoneal administration and conveniently comprise sterile aqueous solutions of the antibody, which are preferably isotonic with the blood of the recipient. Such formulations are typically prepared by dissolving solid active ingredient in water containing physiologically compatible substances such as sodium chloride, glycine, and the like, and having a buffered pH compatible with physiological conditions to produce an aqueous solution, and rendering said solution sterile. These may be prepared in unit or multi-dose containers, for example, sealed ampoules or vials.

The compositions may incorporate a stabilizer, such as for example polyethylene glycol, proteins, saccharides (for example trehalose), amino acids, inorganic acids and admixtures thereof. Stabilizers are used in aqueous solutions at the appropriate concentration and pH. The pH of the aqueous solution is adjusted to be within the range of 5.0-9.0, preferably within the range of 6-8. In formulating the antibody, anti-adsorption agent may be used. Other suitable excipients may typically include an antioxidant such as ascorbic acid.

The compositions may be formulated as controlled release preparations which may be achieved through the use of polymer to complex or absorb the proteins. Appropriate polymers for controlled release formulations include for example polyester, polyamino acids, polyvinyl, pyrrolidone, ethylenevinylacetate, and methylcellulose. Another possible method for controlled release is to incorporate the antibody into particles of a polymeric material such as polyesters, polyamino acids, hydrogels, poly(lactic acid) or ethylene vinylacetate copolymers. Alternatively, instead of incorporating these agents into polymeric particles, it is possible to entrap these materials in microcapsules prepared, for example, by coacervation techniques or by interfacial polymerization, for example, hydroxymethylcellulose or gelatin-microcapsules and poly(methylmethacylate) microcapsules, respectively, or in colloidal drug delivery systems, for example, liposomes, albumin microspheres, microemulsions, nanoparticles, and nanocapsules or in macroemulsions.

The specific antibody of the invention and/or any optional additional agent may be administered systemically, for example, by parenteral routes, such as, intraperitoneal (i.p.), intravenous (i.v.), subcutaneous, or intramuscular routes. The specific antibody of the invention and/or any optional additional agent may be administered systemically, for example, by intraperitoneal (i.p.) route. The specific antibody of the invention and/or any optional additional agent may be administered systemically, for example, by intranasal administration. The specific antibody of the invention and/or any optional additional agent may be administered systemically, for example, by oral administration, by using specific compositions or formulations capable of providing oral bioavailability to proteins. The specific antibody of the invention and/or any optional additional agent may be administered via ocular administration (intraocular). The specific antibody of the invention and/or any optional additional agent may be administered locally, for example, by intracerebroventricular (i.c.v.) administration, for example, by utilizing Alzet minipumps. Antibodies are generally administered in the range of about 0.1 to about 20 mg/kg of patient weight, commonly about 0.5 to about 10 mg/kg, and often about 1 to about 5 mg/kg. In this regard, it is preferred to use antibodies having a circulating half-life of at least 12 hours, preferably at least 4 days, more preferably up to 21 or more days. In some cases it may be advantageous to administer a large loading dose followed by periodic (e.g., weekly) maintenance doses over the treatment period. Antibodies can also be delivered by slow-release delivery systems, pumps, and other known delivery systems for continuous infusion. Dosing regimens may be varied to provide the desired circulating levels of a particular antibody based on its pharmacokinetics. Thus, doses will be calculated so that the desired circulating level of therapeutic agent is maintained.

Typically, the effective dose is determined by the activity of the therapeutic antibody and the condition of the subject, as well as the body weight or surface area of the subject to be treated. The size of the dose and the dosing regime is also determined by the existence, nature, and extent of any adverse side effects that accompany the administration of the antibody and/or the additional agents in the in a particular subject. In determining the effective amount of the therapeutic composition to be administered, the physician needs to evaluate inter alia circulating plasma levels, toxicity, and progression of the disease.

According to some embodiments, there is provided an apoE4 antibody for use in treating or preventing a neurodegenerative condition via systemic administration or systemic route.

According to some embodiments, there is provided a composition comprising an apoE4 antibody and at least one excipient for use in treating or preventing a neurodegenerative condition.

According to some embodiments, there is provided an apoE4 antibody for the preparation of a medicament for treating or preventing a neurodegenerative condition, via systemic route.

According to some embodiments, there is provided a composition comprising an apoE4 antibody and at least one agent capable of modulating expression and/or lipidation of apoE proteins. In some exemplary embodiments, there is provided a composition comprising an apoE4 antibody and Bexarotene.

According to some embodiments, there is provided a composition comprising an apoE4 antibody and at least one agent capable of modulating expression and/or the lipidation of apoE proteins for treating or preventing a neurodegenerative condition.

In some embodiments, the invention provides a kit for treating or preventing a neurodegenerative condition. In some embodiments, the kit comprises a container (such as a vial) comprising an apoE4 antibody in a suitable buffer and instructions for use for systemic administration of the apoE4 antibody. In some embodiments, the kit comprises an injection syringe comprising an apoE4 antibody in a suitable injection buffer.

In various embodiments, when the specific antibody is administered in combination with at least one additional agent, they may be administered according to any of a number of treatment schedules, also referred to “dosing schedules” and “administration regimes”, referring to the frequency of administration and order of administration of each agent.

The terms “comprises”, “comprising”, “includes”, “including”, “having” and their conjugates mean “including but not limited to”. The terms “comprises” and “comprising” are limited in some embodiments to “consists” and “consisting”, respectively.

As used herein the term “about” in reference to a numerical value stated herein is to be understood as the stated value +/−10%.

Having now generally described the invention, the same will be more readily understood through reference to the following examples, which are provided by way of illustration and are not intended to be limiting of the present invention.

### EXAMPLES

### Materials and Methods

Transgenic Mice.

apoE target replacement (TR) mice were created by gene targeting, as previously described (Sullivan, Mezdour et al. 1997, “Targeted replacement of the mouse apolipoprotein E gene with the common human APOE3 allele enhances diet-induced hypercholesterolemia and atherosclerosis.” J Biol Chem 272(29): 17972-80). Construction of these mice differs from other apoE transgenic mice in that the coding region of the mouse apoE sequence was replaced by the corresponding human apoE coding sequences, without affecting the regulatory related portions of the mouse apoE gene. The mice used were purchased from Taconic (Germantown, N.Y.).

Immunohistochemistry and Immunofluorescence Staining.

Mice were anesthetized with ketamine and xylazine and perfused transcardially with saline and then with 4% paraformaldehyde in 0.1M phosphate buffer, pH 7.4. Their brains were removed, fixed overnight in 4% paraformaldehyde in 0.1 M phosphate buffer, pH 7.4, and then placed in 30% sucrose for 48 h. Frozen coronal sections (30 μm) were then cut on a sliding microtome and collected serially. The free-floating sections were immunostained as previously described (Matsumori, Hong et al. 2006, “Enriched environment and spatial learning enhance hippocampal neurogenesis and salvages ischemic penumbra after focal cerebral ischemia.”Neurobiol Dis 22(1): 187-98), with the following primary antibodies (Abs): rabbit anti-Aβ42 (1:500; Chemicon, Temecula, Calif.); rabbit anti 202/205 phospho Tau (1:200, AT-8; Innogenetics); rabbit anti VEGF-A (1:500; Calbiochem); rabbit anti TOM40 (1:500; Santa Cruz); Guinee-pig anti Vglut 1 (1:2000; Millipore); mouse anti GAD67 (1:250; Millipore); mouse anti Synaptophysin (1:200; Sigma); rabbit anti PKCα (1:1000; Santa Cruz); rabbit anti PAX6 (1:400; Covance); sheep anti CHX10 (1:1000; Exalpha biologicals). Sections were washed with 10 mM PBS, pH 7.4, after which the primary antibody, diluted in PBS with 0.1% Triton X-100 (PBST) and with 2% of the appropriate serum, was applied overnight at 4° C. After having rinse in PBST, sections were incubated for 1 hour at room temperature with the secondary antibody (Vector Laboratories, Burlingame, Calif.) and then diluted 1:200 in PBST that contained 2% of the appropriate serum. After several additional rinses in PBST, sections were incubated for 0.5 h in avidin-biotin-horseradish peroxidase complex (ABCElite; Vector Laboratories) in PBST. After rinses in PBST, sections were placed for up to min in diaminobenzidine chromagen solution (Vector Laboratories). The reaction was monitored visually and terminated by rinses with PBS. For the Aβ staining, sections were similarly treated, except that they were pre-incubated before adding the first antibody with 70% formic acid for 7 min. To minimize variability, sections from all animals were stained simultaneously.

Immunofluorescence Staining.

Synaptic markers were evaluated by immunostaining, using fluorescent chromogens. In brief, sections were first blocked by incubation with 0.1% Triton X-100 and 10% normal donkey serum in PBS for 1 h at room temperature. The primary antibodies were then dissolved in 0.1% Triton X-100 and 2% normal donkey serum in PBS, and finally incubated with the sections for 48 h at 4° C. Next, the bound primary antibodies were visualized by incubating the sections for 1 h at room temperature with Alexa-fluor 488-conjugated donkey anti-rabbit (1:1000; Invitrogen, Eugene, Oreg.), Alexa-fluor 488-25 conjugated donkey anti-mouse (1:1000; Invitrogen), or Alexa-fluor 488-conjugated goat anti Guinee-pig (1:1000; Invitrogen), depending on the appropriate initial antibodies. The sections were then mounted on dry gelatin-coated slides, and fluorescence was visualized using a confocal scanning laser microscope (LSM 510; Zeiss, Oberkochen, Germany). Images (1024×1024 pixels 12 bit) were obtained by a 20× lens averaging eight scans per slice.

Image Analysis.

The peroxidase-immunostained sections were viewed and photographed with a 40× objective and a Nikon DS-5M camera (Nikon Instech, Tokyo, Japan). The intensities of immunohistochemical staining as the percentage area stained were determined with the aid of the Image-Pro Plus system (version 5.1; Media Cybernetics, Silver Spring, Md.). Other than making moderate adjustments for contrast and brightness, the images were not manipulated. Images were taken at 10× from the dentate gyrus (DG), CA3 and CA1 (hippocampal subfields 3 and 1), respectively. The percent of stained area (over a threshold) was calculated for each area, and the results were normalized to the levels of the ApoE3 group.

Immunoblot Analysis.

Immunoblot analysis was performed as described (Haas, Liraz et al. 2012, “The Effects of Apolipoproteins E3 and E4 on the Transforming Growth Factor-beta System in Targeted Replacement Mice”. Neurodegener Dis 10(1-4): 41-5). In short, mice were trans-cardially perfused with PBS and then decapitated. After which, their brain were processed in 2 inter-changeable procedures: 1) the hippocampus was rapidly excised and frozen in liquid nitrogen. 2) the intact brains were rapidly frozen in liquid nitrogen, after which the hippocampus was isolated from the frozen brain by cutting it into 500-μm coronal slices utilizing a frozen mold, from which the hippocampi (Bregma −3 to −2) was excised surgically. The hippocampi were homogenized in 180 μl homogenization buffer (10 mM HEPES, 2 mM EDTA, 2 mM EGTA, 0.5 mM DTT, protease inhibitor cocktail (Sigma P8340) and phosphatase inhibitor cocktail (Sigma P5726)), after which the samples were aliquoted and stored at −70° C. Protein concentration was determined utilizing the BCA protein assay kit (Pierce 23225). Gel electrophoresis and immunoblot assays were performed for the following antibodies: Mouse anti Vglut1 (1:1000; Millipore); Antigen levels were visualized utilizing the ECL chemiluminescent substrate (Pierce), after which the intensity of the immunoblot bands was quantified using EZQuantGel software (EZQuant, Tel Aviv, Israel).

Reverse Transcription (RT)-PCR Analysis.

The animals were sacrificed at the age of 3-4 months and intracadial perfused with saline. Their hippocampi were removed. The samples were kept in −70° c. RNA was extracted from the tissue using the MasterPure RNA purification kit (Epicentre, USA). RNA was transformed into cDNA using High Capacity cDNA reverse transcription kit (Applied Biosystems, USA). TaqMan qRT PCR assays were conducted according to the manufacturer's specifications (Applied Biosystems). Oligonucleotides (probes) for TaqMan qRT PCR were attached to FAM (6-carboxyfluorescin) at the 5′ end and a quencher dye in the 3′ end. Gene expression levels were determined utilizing TaqMan RT-PCR specific primers (primers used are TaqMan® Gene Expression Assays, Applied Biosystems, CA, USA). Analysis and quantification were conducted in comparison to the expression of the Hprt-1 house keeping gene.

Behavioral Experiments.

The spatial navigation test was performed by a dry maze modification of the hole board test (Van der Zee, Compaan et al. 1992, “Changes in PKC gamma immunoreactivity in mouse hippocampus induced by spatial discrimination learning.” J Neurosci 12(12): 4808-15), which monitors the ability of the mice to locate a small water-filled well in a circular arena.

The mice were water deprived for Two days before the experiment and throughout the entire experiment, the mice were subjected to a 23 h/day water deprivation regime, in which mice were able to drink ad libitum for 1 h every day. Following those 2 days mice were placed in a circular arena (95 cm diameter, with 20 evenly separated wells; 1 cm depth, 0.5 cm diameter) in which all the wells were filled with 100 μl of water. Every mouse then underwent four runs per day for 2 d; each run consisted of 120 s. Mice were allowed to drink from all the wells they could locate during these runs. The arena was cleaned with 70% ethanol between every run. Following habituation, the mice were subjected to the arena for 4 runs a day. Each run lasted up to 120 s and only one well was filled with water. If the mouse found the water-filled well, then it was allowed to drink for 15 s, whereas if the mouse did not find the well, the mouse was brought to it after 120 s and allowed to remain there for 15 s. This was performed for 8 days. To elevate the level of complexity of the test, the location of the water-filled well was then changed to a new location on day 9, and the performance of the mice was then tested in this configuration for five more days. Latency to the water-filled well was measured for each trial.

Retinal Sections Preparation:

Following mice decapitation, eyes were enucleated and placed in fresh PFA 4% for 1 h. Next, the lens was removed and the remaining eye cup was placed again in fresh PFA 4% for 1 h. The eye cups were then washed with PBS and placed in 15% sucrose for 1 h following 30% sucrose over night. The eye cups were then placed in freezing medium for 45 minutes and frozen in dry ice. 16 μm thin sections of the eyes were sectioned with cryostat and placed on glass slides.

Retinal Sections Staining:

Slides were placed in PFA 4% for min, washed in PBS and blocked in PBStg (triton 0.2% and gelating 0.2%) for 2 h in RT. Then washed ×3 in PBStg and ×3 in PBS and placed with 1^{st} antibody ON in 4° c. The next day the slides were washed ×3 in PBStg and ×3 in PBS and incubated with 2^{nd} antibody 2 h at RT, then washed again ×3 in PBStg and ×3 in PBS and mounted with mounting medium.

Preparation of mAbs which React Specifically with apoE4.

ApoE-deficient mice were immunized with an apoE-derived peptide, which contains the amino acid sequence specific to apoE4 (i.e. Arg at position 112). Hybridomas were than screened by several complimentary methods for production of apoE4-specific mAb's. These include immunoprecipitation, for selection of mAbs that bind native apoE4 in solution, western blotting and ELISA for quantifying apoE4, and immunohistochemistry, for localization of apoE4. Such exemplary monoclonal antibody, is the mAb 9D11 (Covance, Cat. No. SIG-39754).

I.C.V. (Intracerebroventricular) Injections.

The apoE4 mAb (0.5 mg/ml) was injected i.c.v. together with the neprilyn inhibitor thiorphan for one week utilizing Alzet miniosmotic pumps.

I.P (Intraperitoneal) Injections.

ApoE TR mice which were i.p injected with either the 9D11 apoE4 mAb (cat# SIG-39754; Covance) or with control IgG1 mAb (MOPC-21, cat #BE0083-100; Bio X-cell,) (10 mk/kg weekly for weeks following weaning), as well as corresponding sham mice which were similarly treated with PBS.

Statistical Analysis.

Results (mean±SE) are expressed as percentages of the levels of ApoE3 TR mice. Student's T-test was performed between the ApoE3 and ApoE4 groups. Bonferroni correction was employed for multiple comparisons when needed.

### Example 1-13

### Characterization of ApoE4 Phenotypes in Young Targeted Replacement Mice

### Example 1

### Effects of ApoE Genotype on Performance in the Dry Version of the Morris Water Maze of Young ApoE3 and ApoE4 TR Mice

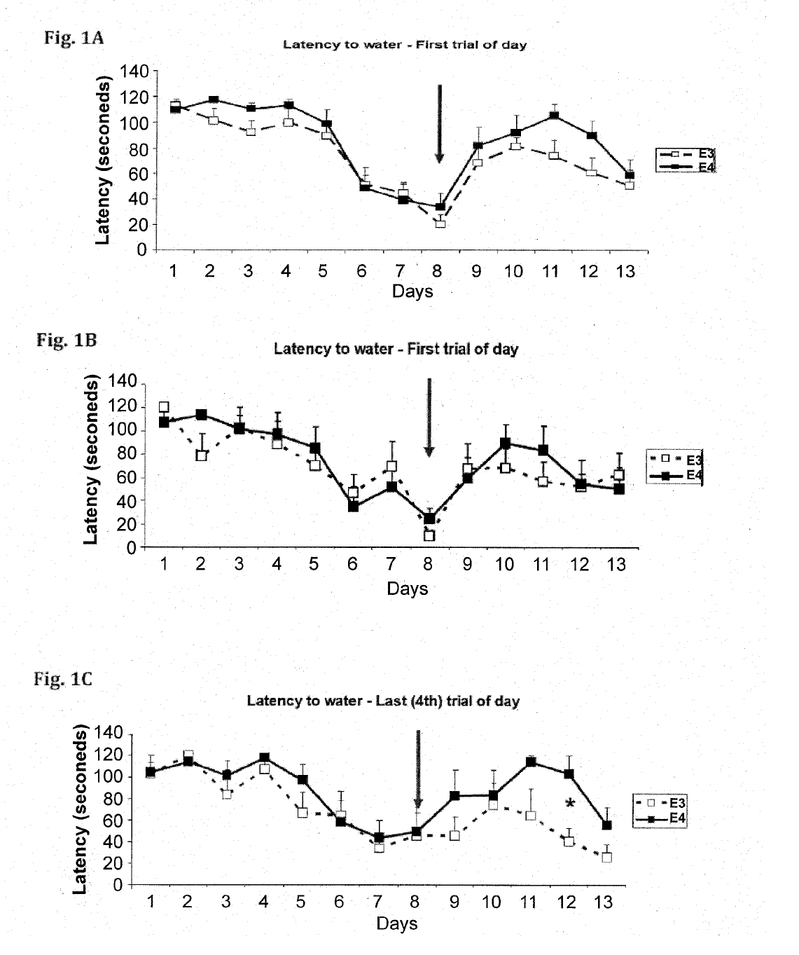

Naïve ApoE3 and apoE4 mice, at 4 months of age, were subjected to the dry version of the Morris Water Maze, as detailed above. The mice were subjected to 4 daily trials and the latency to the water filled well was measured. The experiment was performed in 2 phases, such that after the 8^{th} day, the location of the water filled well was altered. The results are presented in FIGS. 1A-C. As can be seen, the results indicate that the performance of the apoE4 mice (i.e. the latency to reach the platform) was worse (i.e. longer) than that of the apoE3 mice during the second phase of experiment, namely after the position of the water filled well, was changed. Furthermore, this effect was most pronounced during the last trial of each day. These findings suggest that the apoE4 mice are impaired in their ability to relearn an altered task in the dry maze.

### Example 2

### Effects of ApoE Genotype on Vglut1 Immuno-Reactivity in the Hippocampus of Human ApoE Targeted Replacement Mice

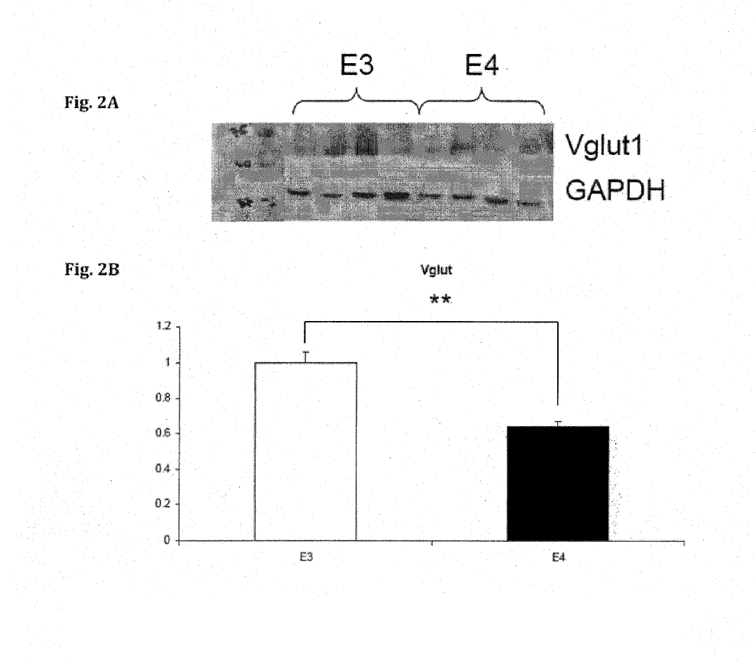

Naïve ApoE3 and ApoE4, mice, at 4 months of age, were sacrificed and their brains extracts subjected to Western Blot analysis, as detailed above. Western Blots of samples of whole hippocampi from ApoE3 and ApoE4 mice were immunostained with anti-Vglut antibody (n=11). The results are presented in FIGS. 2A-B. As can be seen, the results indicate that apoE4 lowers the levels of the presynaptic glutamate transporter Vglut1 of hippocampal glutamatergic neurons.

### Example 3

### Effects of ApoE Genotype on Vglut1 Immuno-Reactivity in the Hippocampus of Human ApoE Targeted Replacement Mice

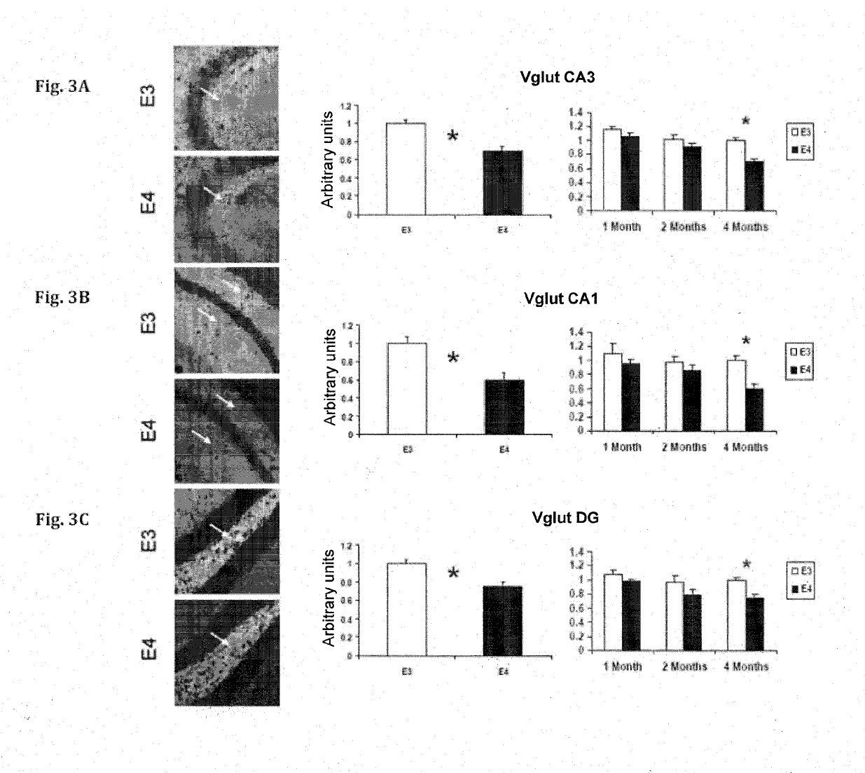

Naïve 4 months old ApoE3 and ApoE4 brain slices were immunostained with anti-Vglut1 antibody and the expression of Vglut1 was tested. The results are presented in FIGS. 3A-C. As can be seen, the results indicate that apoE4 lowers the levels Vglut1 of CA1 CA3 and DG hippocampal subfields.

### Example 4

### Effects of ApoE Genotype on Aβ42 Immuno-Reactivity in the Hippocampus of Human ApoE Targeted Replacement Mice

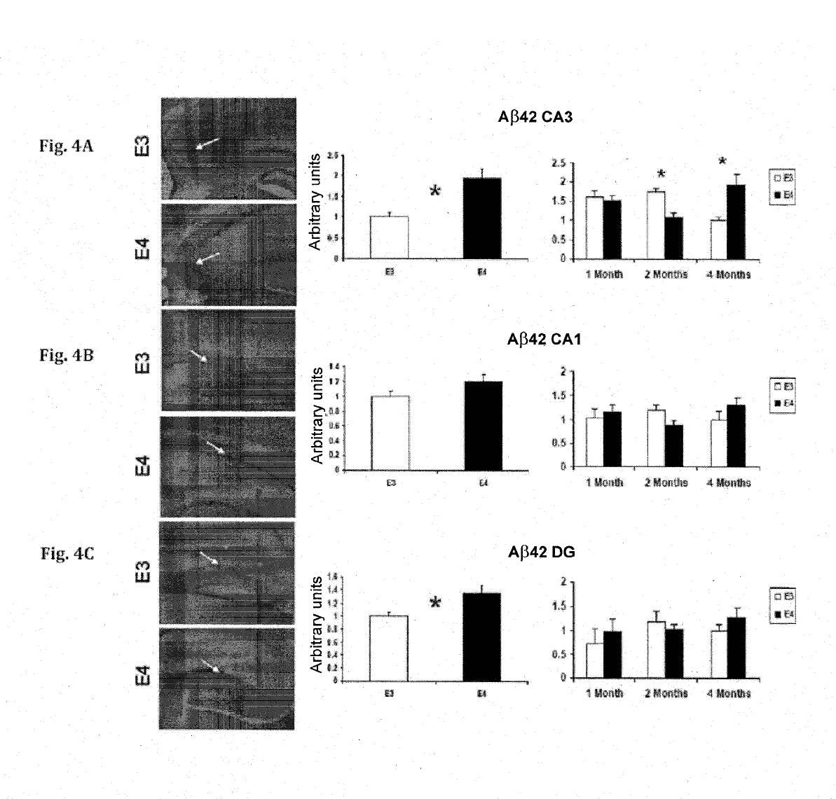

Naïve 4 months old ApoE3 and ApoE4 brain sections were immunostained with anti-Aβ42 antibody, as detailed above. The expression of the 40-42 amino acid long amyloid beta (Aβ) peptide was determined in various regions. The results are presented in FIGS. 4A-C. As can be seen, the results indicate that apoE4 induces the accumulation of Aβ42 is hippocampal neurons and that this effect is most pronounced in CA3 neurons.

### Example 5

### Effects of ApoE Genotype on AT-8 Immuno-Reactivity in the Hippocampus of Human ApoE Targeted Replacement Mice

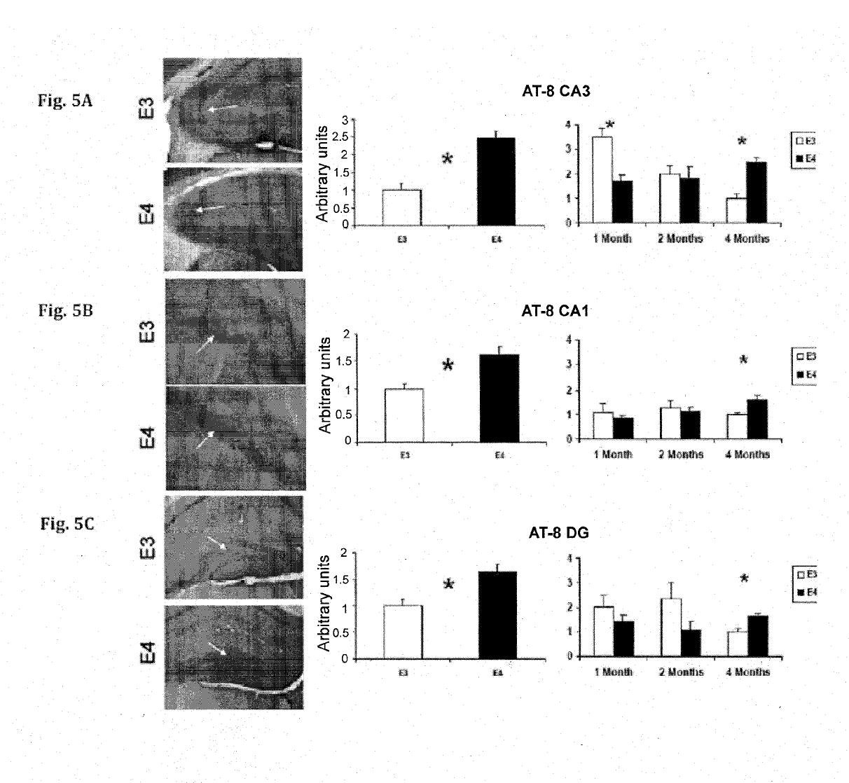

Naïve 4 months old ApoE3 and ApoE4 brains were immunostained with anti-202/205 phospho Tau antibody (AT-8), as detailed above. The presence of phosphorylated Tau was determined in various brain regions. The results are presented in FIGS. 5A-C. As can be seen, the results indicate that apoE4 triggers the accumulation of phosphorylated tau in hippocampal neurons and that this effect is most pronounced in CA3 neurons.

### Example 6

### Effects of ApoE Genotype on VEGF Immuno-Reactivity in Different Brain Regions of Human ApoE Targeted Replacement Mice

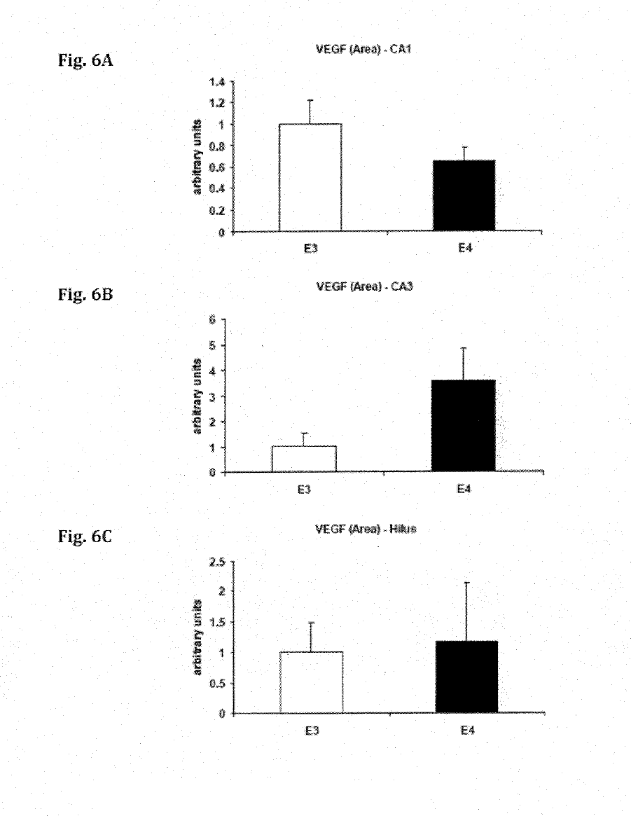

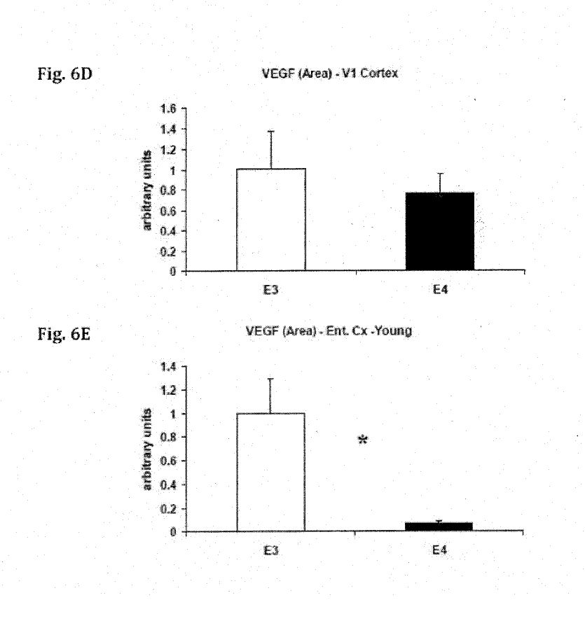

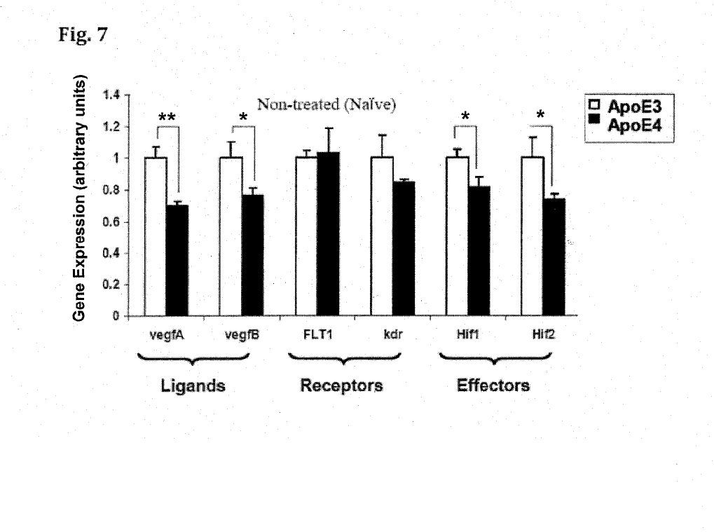

Naïve 4 months old ApoE3 and ApoE4 brain slices were immunostained with anti-VEGF antibody, as detailed above. The expression of VEGF was determined in various brain regions of the mice. The results are presented in FIGS. 6A-E. As can be seen, the results indicate that the VEGF levels are significantly lower in the enthorhinal cortex of the apoE4 compared to the apoE3 young mice.

### Example 7

### The Effects of apoE Genotype on the Basal Gene Expression Levels of HIF Transcription Factors and VEGF Ligands and Receptors

RT-PCR analysis was performed on mRNA extracted from the hippocampus of ApoE3 and apoE4 targeted replacement non-treated (Naïve) mice. The RT-PCR analysis was performed as described above, with specific primers for Hif1, Hif2, VegfA, VegfB, FLT1 or kdr (primers used are TaqMan® Gene Expression Assays, Applied Biosystems, CA, USA). Relative expression levels (arbitrary units) of the various tested genes in the hippocampus of ApoE3 or ApoE4 mice are represented in the bar graph shown in FIG. 7. As can be seen, the results indicate that the expression levels of both VEGF ligands (e.g. VegfA and VegfB) and transcription factors (e.g. Hif1 and Hif2) are significantly decreased in apoE4 compared to apoE3 young mice, whereas the VEGF receptors expression levels remain unchanged.

### Example 8

### Effects of ApoE Genotype on TOM40 and COX1 in the CA3 of the Hippocampus of Human ApoE Targeted Replacement Mice

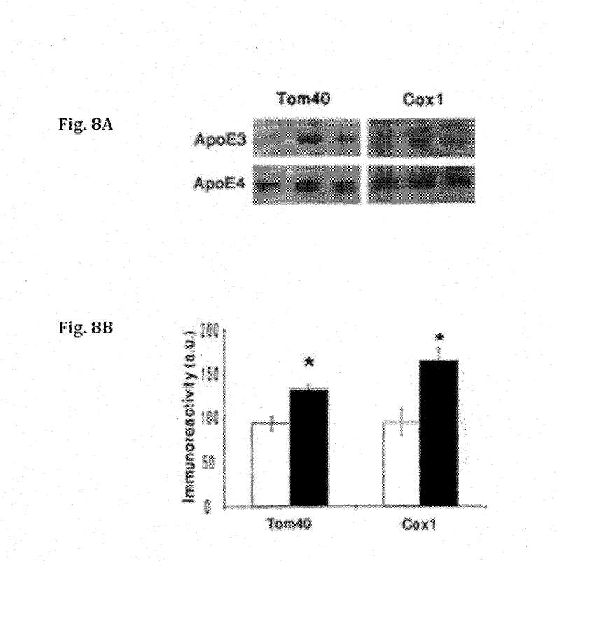

Naïve ApoE3 and ApoE4, mice, at 4 months of age, were sacrificed and their brains extracts were subjected to Western Blot analysis, as detailed above. Western Blots of samples of whole hippocampi from ApoE3 and ApoE4 mice were immunostained with anti-TOM40 or anti-COX1 antibodies. The expression of TOM40 and COX1 was determined. The results are shown in FIGS. 8A-B. As can be seen, the results indicate that the protein level of both COX1 and TOM40 are higher in the apoE4 mice than the apoE3 mice.

### Example 9

### Effects of ApoE Genotype on TOM40 Immuno-Reactivity in the CA3 of the Hippocampus of ApoE Targeted Replacement Mice

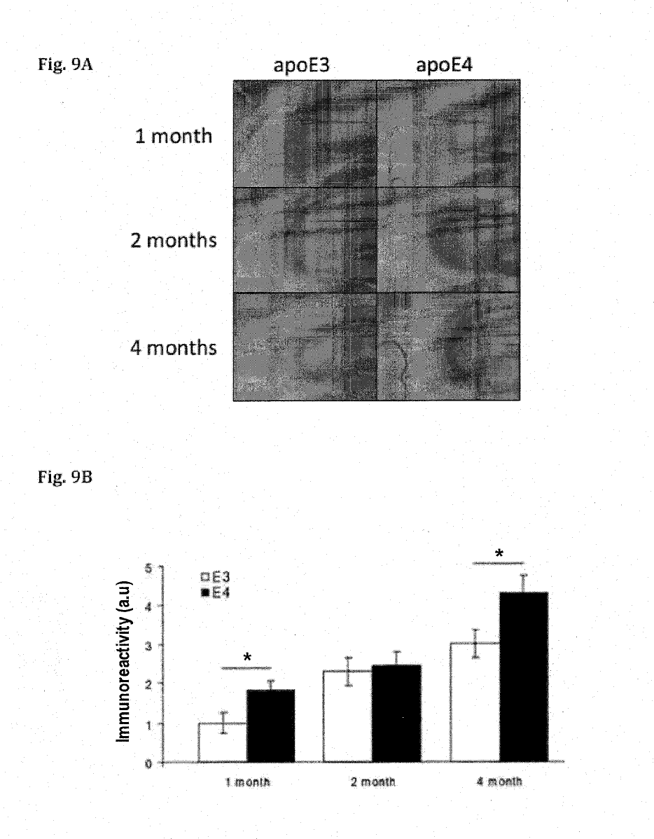

Brain sections of Naïve 1, 2 and 4 months old ApoE3 and ApoE4 mice prepared and immunostained, as described above, with anti-TOM40 antibody, to determine expression of the TOM40 protein expression. The results presented in FIGS. 9A-B show the expression of TOM40 protein in CA3 region of the ApoE3 and ApoE4 mice. As can be seen, the results indicate that the levels of TOM40 increase with age in both the apoE3 and the apoE4 mice. However, there are higher levels of TOM40 in the apoE4 mice in both 1 month of age and 4 months of age.

### Example 10

### Effects of ApoE Genotype on ApoE Receptors in the CA3 and CA1 of Hippocampus of Human ApoE Targeted Replacement Mice

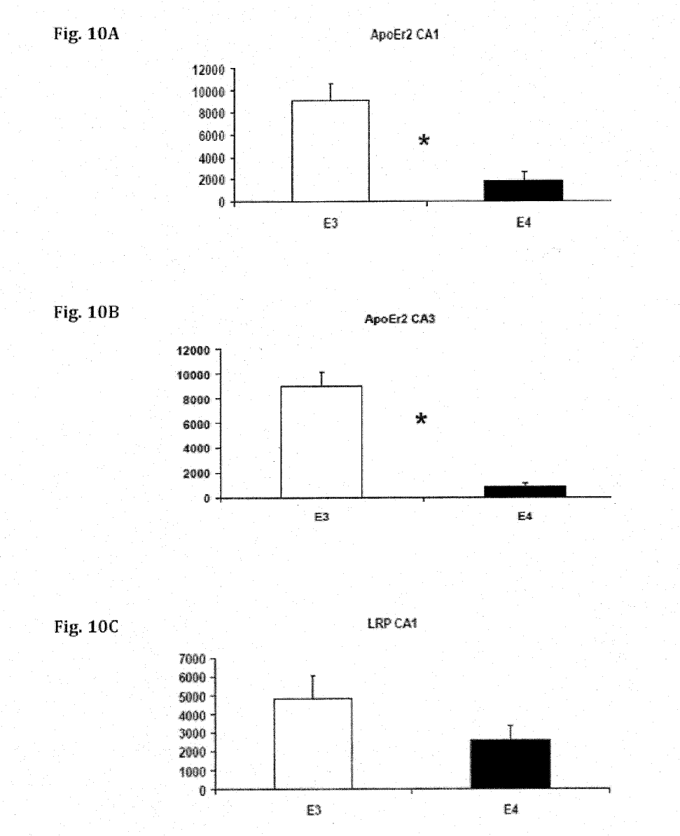

Brains from Naïve 4 months old ApoE3 and ApoE4 were immunostained with anti-LRP and anti-ApoEr2 antibodies. LRP is a classical endocytosis receptor, mediating the entrance of apoE, APP, Aβ and many other ligands and ApoEr2 is a receptor that mediates signaling processes. The results showing the expression of ApoEr2 in CA1 and CA3 are shown in FIGS. 10A-B, and the expression of LRP is shown in CA1 region. Altogether, the results indicate that ApoEr2 is markedly down regulated in both CA1 and CA3 neurons of apoE4 mice, while LRP is only slightly down regulated in the CA1 of ApoE4 mice.

### Example 11

### Effects of ApoE Genotype on Mitochondrial and Lysosomal Ultrastructure in Hippocampus of Young (3 Months Old) apoE4 Mice

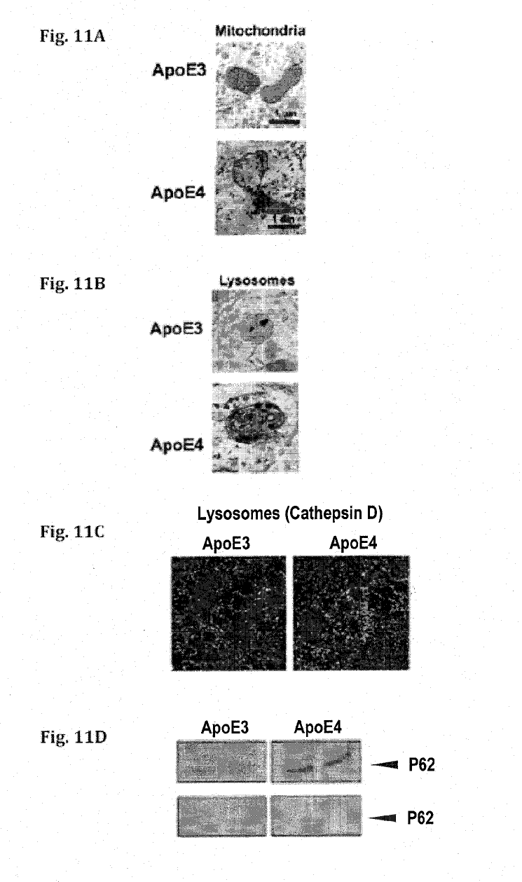

Electron microscopy images of cells showing mitochondrial ultrastructure in ApoE3 (top panel) or ApoE4 (lower panel) mice are shown in FIG. 11A. FIG. 11B shows confocal microscopy images of cells demonstrating lysosomal ultrastructure in ApoE3 (top panel) or ApoE4 (lower panel) mice. FIG. 11C shows Pictograms of immunohistochemical analysis of expression of Cathepsin D in hippocampus of apoE3 or apoE4 mice. FIG. 11D shows pictograms showing immunoblots of expression of the autophagy substrate P62 in ApoE3 or ApoE4 mice. The results indicate that, as can be seen in FIG. 11A, expression of ApoE4 is associated with mitochondrial morphological aberrations. Additionally, the EM and cathepsin-D confocal microscopy images shown in FIG. 11B-C reveal lysosomal enlargement, which is further associated with elevated levels of the lysosomal-autophagy substrate P62 in the CA3 subfield of the apoE4 mice, suggesting that that protein degradation via this pathway is impaired by apoE4. Thus, the results suggest that the mitochondria and the autophagy-lysosomal pathway are already affected by apoE4 at a young age. The results further provide appropriate markers for assessing the efficacy of treatment with apoE4 mAbs.

### Example 12

### The Effect apoE Genotype on Synaptic Parameters in the Inner Plexiform Layer (IPL) of the Retina

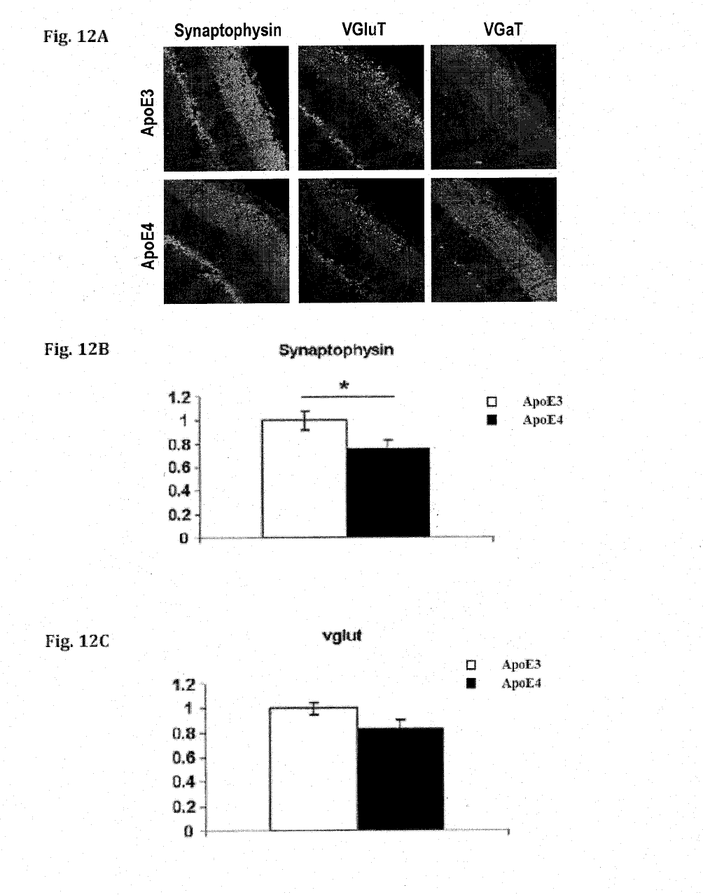

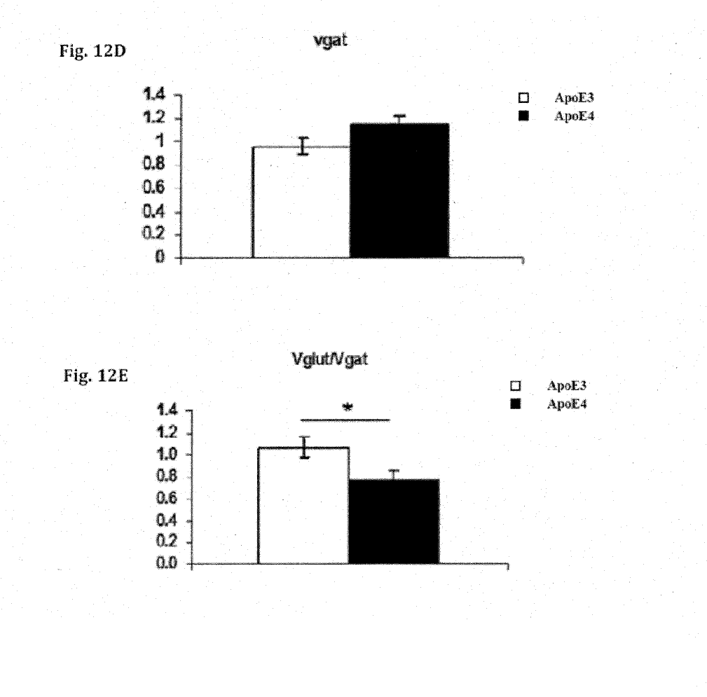

Cross sections of apoE3 and apoE4 retinas were stained with antibodies against Synaptophysin (total synapses), Vglut (marker for excitatory synapses) and Vgat (marker for inhibitory synapses). The expression of these proteins is shown in FIGS. 12A-E. As can be seen, the results indicate that reduced levels of synapses in the IPL accompanied by reduced excitatory activity in the IPL of the apoE4 compared to apoE3 retinas.

### Example 13

### The Effect of apoE Genotype on Neuronal Parameters in the Retina

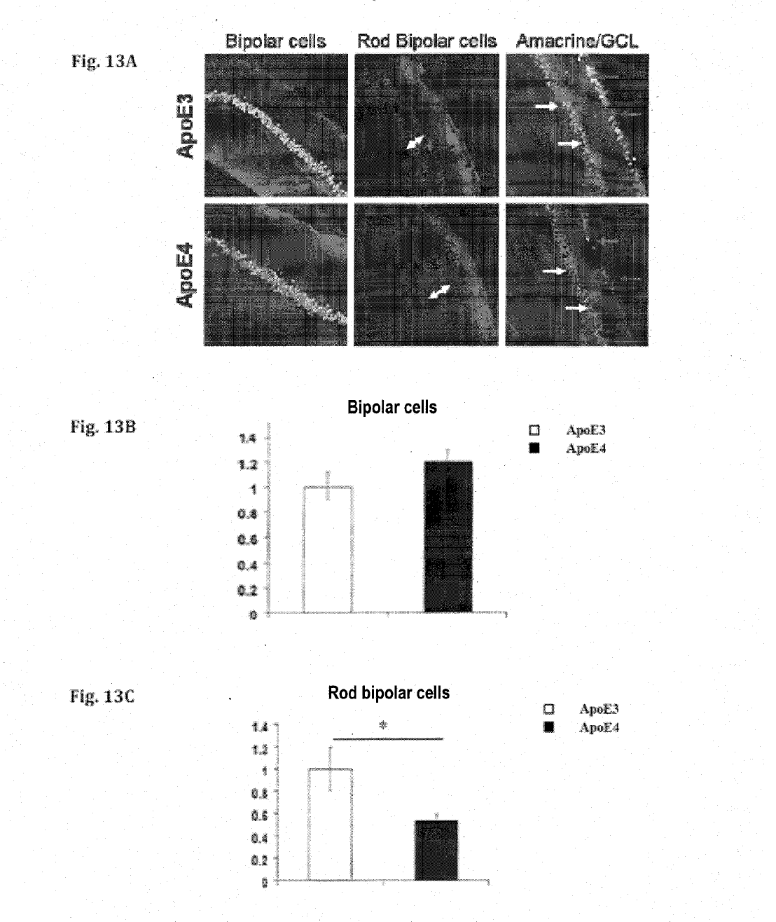

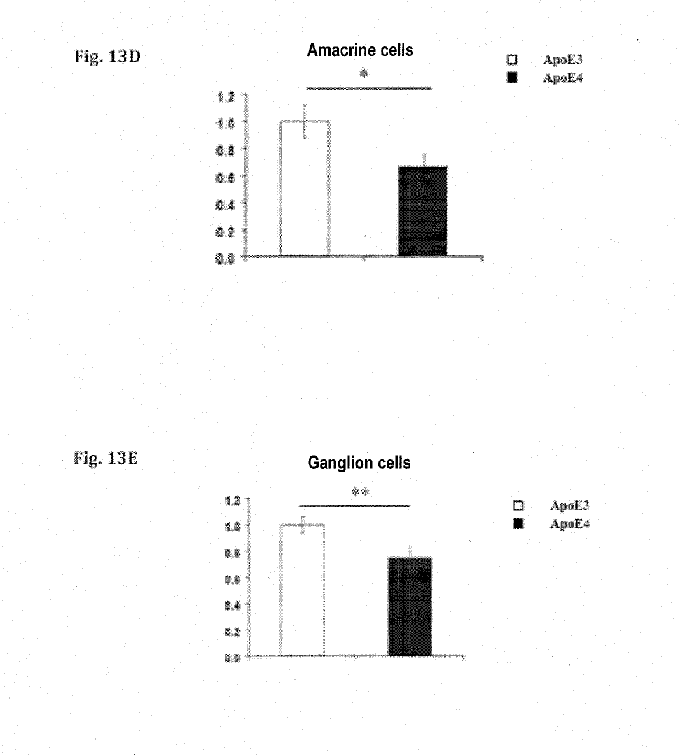

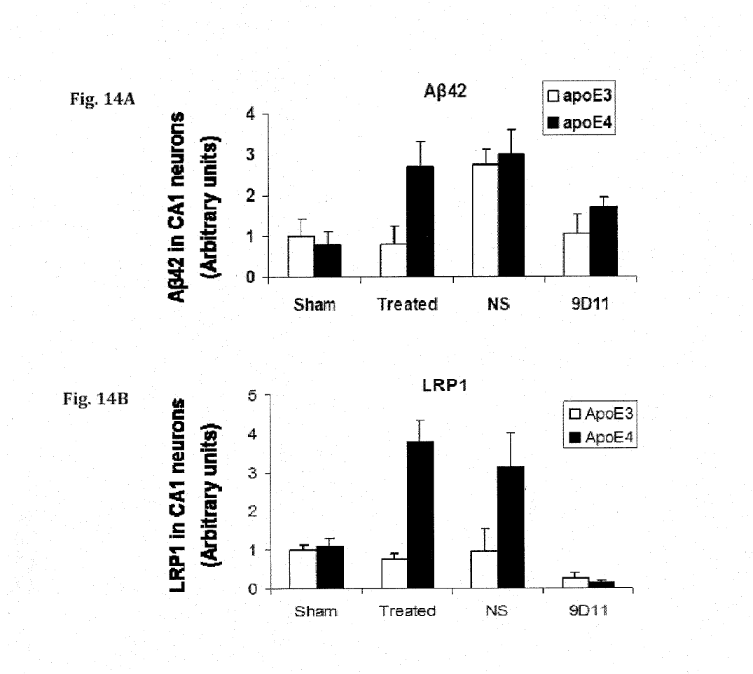

Cross section of apoE3 and apoE4 retinas were stained for bipolar cells (with anti-αCHX10 antibody), Rod bipolar cells (with anti-αPKCα), amacrine and Ganglion cells (GCL) with PAX6 as a marker. The results are presented in the pictograms shown in FIGS. 13A-E. As can be seen, the results indicate that that apoE has isoform specific effect on Rod bipolar, amacrine and ganglion cells of the retina while total bipolar cells seem to be unaffected.

### Examples 14-19

### Effects of apoE4 Monoclonal Antibodies on Young Targeted Replacement Mice, as Preventative Paradigm or Treatment Paradigm

### Example 14

### Direct i.c.v Injection of apoE4 Monoclonal Antibodies

It has been previously shown that i.c.v injection of the Aβ degrading enzyme neprilysin results in the apoE4 accumulation of Aβ in hippocampal neurons, which in turn triggers the degeneration of the affected neurons and subsequent cognitive impairments (Belinson H, Lev D, Masliah E, and Michalson D M (2008) J. Neuroscience 28, p. 4690). These effects are associated with upregulation of the apoE receptor, LRP, in hippocampal neurons of the affected hippocampal neurons and with a parallel decrease in the levels of these receptors in the corresponding hippocampal neurons of the apoE3 mice. It has also been shown (Gal Ophir PhD Thesis, Tel Aviv University, Israel, 2006), that specific anti apoE4 antibodies can be administered directly to the brain via i.c.v. infusion and can reduce apoE4-induced generation of brain Aβ deposits following the inhibition of an AP-degrading enzyme.

The experiment provided herein was directed at assessing the extent to which the apoE4-Aβ driven elevation in LRP levels and accumulation of Aβ can be prevented by i.c.v. injection of the apoE4 mAb (9D11). The apoE4 mAb (0.5 mg ml) was injected (i.c.v.) together with the neprilysin inhibitor thiorphan (0.5 mM; Sigma T6031), for one week utilizing Alzet miniosmotic pumps, Sham treated mice were i.c.v. injected with ACSF and no IgG nor thiorphan. The “thiorphan” mice were similarly treated except that they were injected with ACSF containing thiorphan. The non specific and apoE4 IgG groups were similarly treated except that they were injected with thiorphan together with either the non specific IgG, or apoE4 antibody. The results, shown in FIGS. 14A-B demonstrate that apoE4 mAbs administrated by the i.c.v. route, prevented the apoE4 driven accumulation of Aβ in hippocampal neurons and the associated increase in the levels of the apoE receptor LRP. These results may serve as a proof of concept that the apoE4 antibody can counteract apoE4 related effects when inserted directly into the brain.

### Examples 15-18

### Preventive Effect of Intraperitoneal (i.p.) Injection of apoE4 Monoclonal Antibody on Young Targeted Replacement Mice

Naïve apoE3, and apoE4 homozygote mice were weekly administered by i.p. injection with: PBS injection, injection of non-specific monoclonal antibody (mAb, MOPC-21, Bio X-Cell), or apoE4 monoclonal antibody (9D11, 10 mg/kg) for 3 months (10 weeks). At the age of four months, the mice were sacrificed. Brain slices of the mice were subjected to biochemical and immunohistochemical analysis, as detailed below, in each of the specific examples (Examples 15-17 and 19). In addition, behavioral testing was performed on a different group of similarly treated mice (Example 18).

### Example 15

### Preventive Effect of Intraperitoneal (i.p.) Injection of apoE4 Monoclonal Antibody on Phosphorylated Tau in the Hippocampus of TR Mice

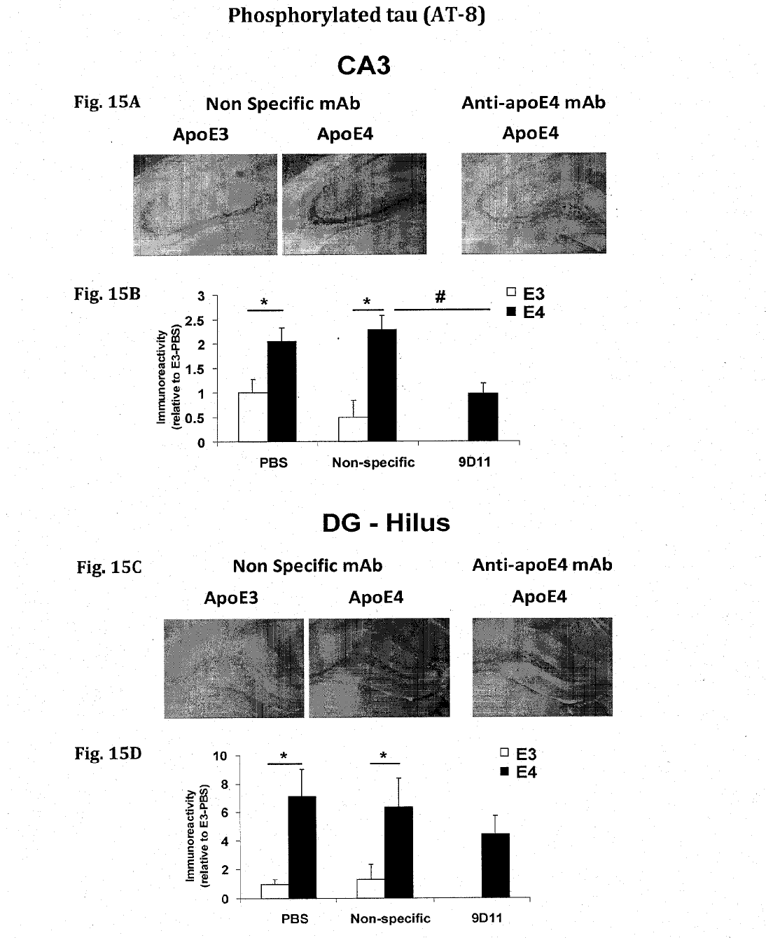

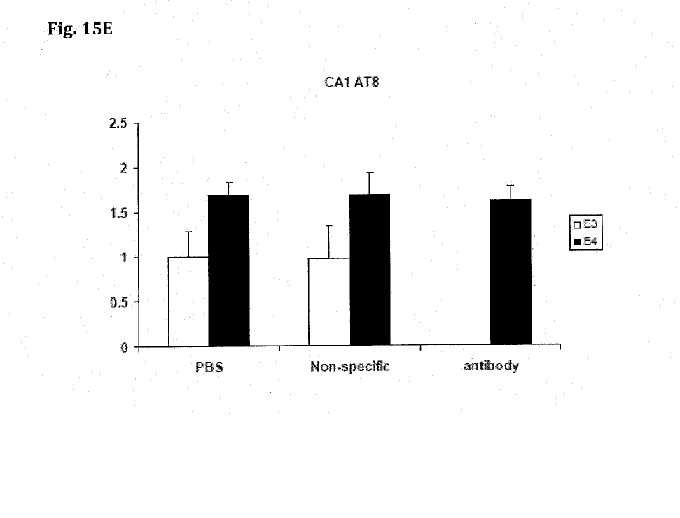

Immunohistochemical analysis using AT-8 antibody, directed against phosphorylated tau (n=4-6) was preformed. The results presented in FIG. 15A, show representative AT8 stained coronal sections of the CA3 subfield of apoE3 and apoE4 mice treated with non specific IgG and of apoE4 mice treated with apoE4 mAb. The results presented in FIG. 15B show and bar graph of quantification of expression of AT-8 in the CA3 region. The results presented in FIG. 15C, show representative AT8 stained hippocampal sections of the DG-hilus region of apoE3 and apoE4 mice treated with non specific IgG and of apoE4 mice treated with apoE4 mAb. The results presented in FIG. 15D show bar graph of quantification of expression of AT-8 in the DG region. The results presented in FIG. 15E show bar graph of quantification of expression of AT-8 in the CA1 region. The results are displayed as mean+/− SE of expression of AT-8 at various brain regions of E3 or E4 mice, under the indicated treatments. White bars correspond to ApoE3 mice, and black bars correspond to ApoE4 mice. * indicate P<0.05 between ApoE3 and ApoE4, # indicate p<0.05 between ApoE4 mice injected with non-specific mAb and the specific anti-apoE3 monoclonal antibody (9D11). The results are normalized to the PBS injected ApoE3 mice. As can be seen, the results indicate that the tau hyperphosphorylation induced by apoE4 in hippocampal CA3 neurons is blocked by i.p. injection of anti-apoE4 mAbs.

### Example 16

### Preventive Effects of Intraperitoneal (i.p.) Injection of apoE4 Monoclonal Antibody on Aβ42 or Vglut1 in the Hippocampus of TR Mice

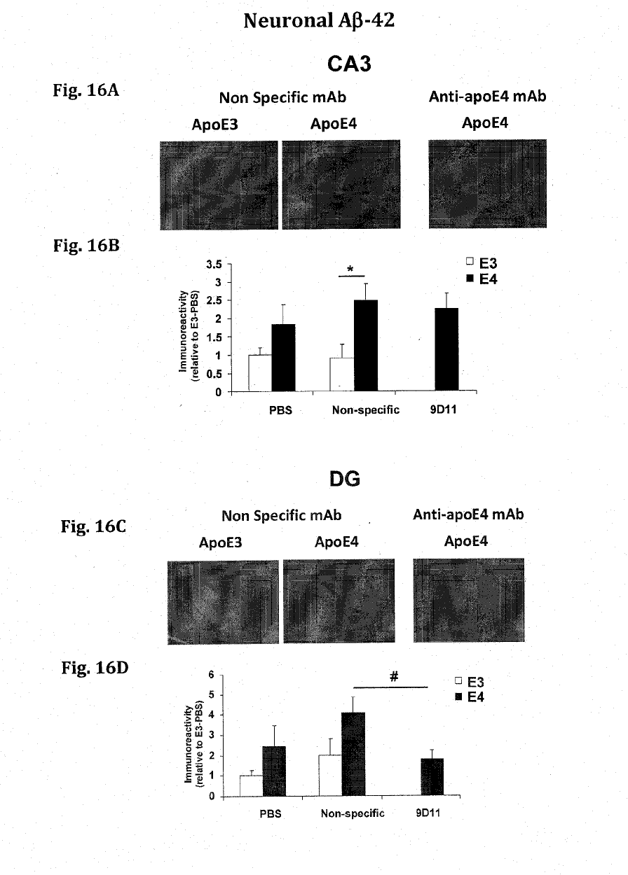

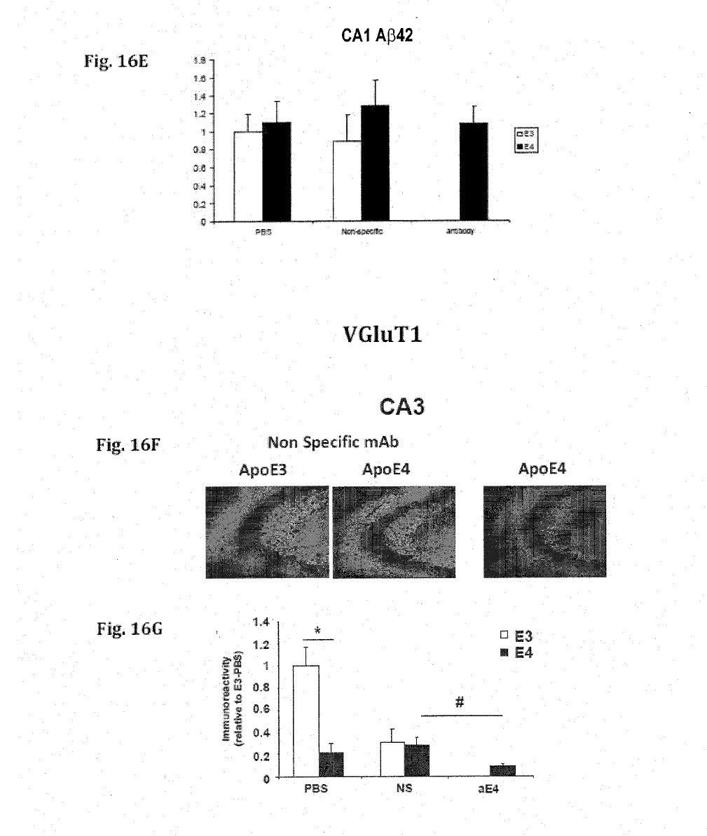

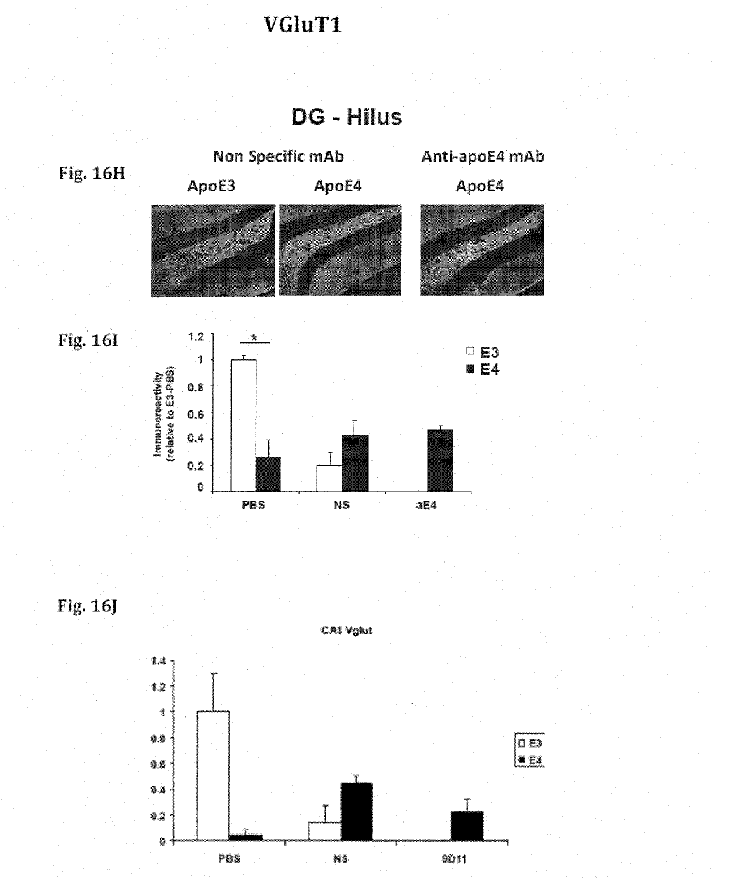

Immunohistochemical analysis using anti-Aβ42 or anti-Vglut1 antibodies (n=4-6) were performed on brain slices of the mice. Results presented in FIG. 16A, show representative pictograms of Aβ42 stained (mAb G2-11) coronal sections of the CA3 subfield of apoE3 and apoE4 mice treated with non specific IgG and of apoE4 mice treated with the apoE4 mAb. The results shown in FIG. 16B show bar graph of quantification of expression of Aβ42 in CA3. Results presented in FIG. 16C, show representative pictograms of Aβ42 stained (mAb G2-11) coronal sections of the DG subfield of apoE3 and apoE4 mice treated with non specific IgG and of apoE4 mice treated with the apoE4 mAb. The results shown in FIG. 16D show bar graph of quantification of expression of Aβ42 in DG. The results shown in FIG. 16E show bar graph of quantification of expression of Aβ42 in CA1. The results presented in FIG. 16F, show representative pictograms of VGluT1 stained sections of the CA3 subfield of apoE3 and apoE4 mice treated with non specific IgG and of apoE4 mice treated with the apoE4 mAb. The results shown in FIG. 16G show bar graph of quantification of expression of VGluT1 in CA3. The results presented in FIG. 16H, show representative pictograms of VGluT1 stained sections of the DG-Hilus subfield of apoE3 and apoE4 mice treated with non specific IgG and of apoE4 mice treated with the apoE4 mAb. The results shown in FIG. 16I show bar graph of quantification of expression of VGluT1 in DG. The results shown in FIG. 16J show bar graph of quantification of expression of VGluT1 in CA1. The results are displayed as mean+/− SE of expression of Aβ42 or Vglut1 at the various brain regions of E3 or E4 mice, under the indicated treatments. White bars correspond to ApoE3 mice, and black bars correspond to ApoE4 mice. * indicate P<0.05 between ApoE3 and ApoE4 mice. All results are normalized to the PBS injected ApoE3 mice. These result show that, Aβ42 and VGlut1 related phenotypes of apoE4 are mildly affected by i.p. injection of the apoE4-mAbs.

### Example 17

### Preventive Effects of Intraperitoneal (i.p.) Injection of apoE4 Monoclonal Antibody on apoER2 Receptor in TR Mice

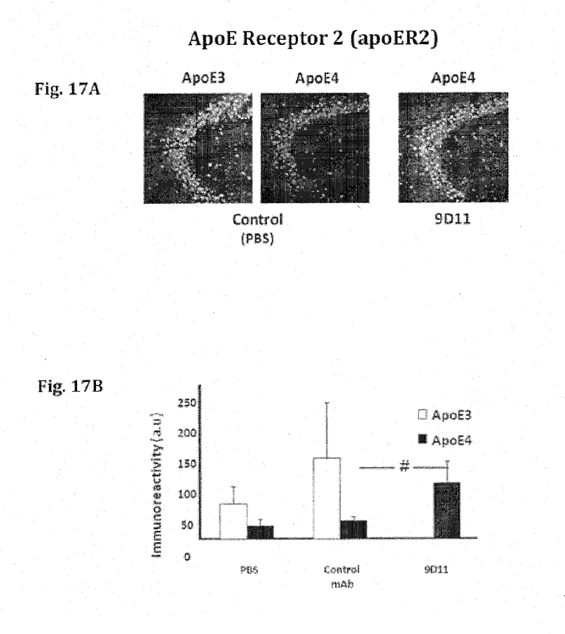

Immunohistochemical analysis of ApoE receptor 2 (ApoER2), using anti-ApoER2 (n=4-6) was performed on brain slices of the mice. The analysis was performed in the CA3 hippocampal subregion of 4 months old apoE3 or apoE4 mice. The results, presented in FIG. 5 17A, show representative pictograms of coronal sections of the CA3 subfield of apoE3 and apoE4 mice, treated with non specific IgG or apoE4 mice treated with the apoE4 mAb, each stained with anti-ApoER2 antibody. The results shown in FIG. 17B show bar graph of quantification of expression of ApoER2 receptor in CA3. White bars correspond to ApoE3 mice, and black bars correspond to ApoE4 mice. # indicate P<0.05 between ApoE3 and ApoE4 mice. All results are normalized to the PBS injected ApoE3 mice. These result show that the apoER2 decrease, driven by apoE4, can be reversed (prevented) by i.p. injection of the apoE4-mAbs.

### Example 18

### Preventive Effects of Intraperitoneal (i.p.) Injection of apoE4 Monoclonal Antibody on Cognitive Impairments of TR Mice

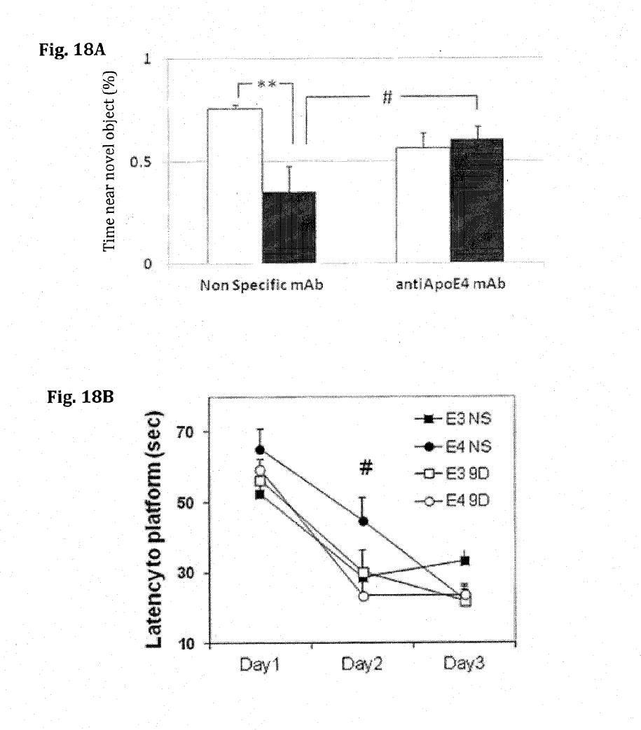

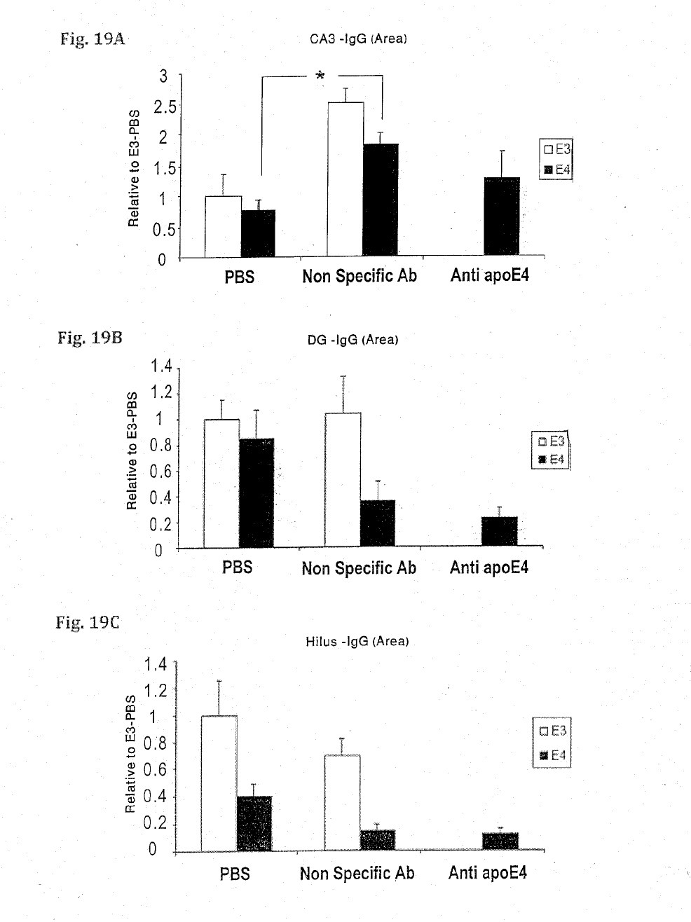

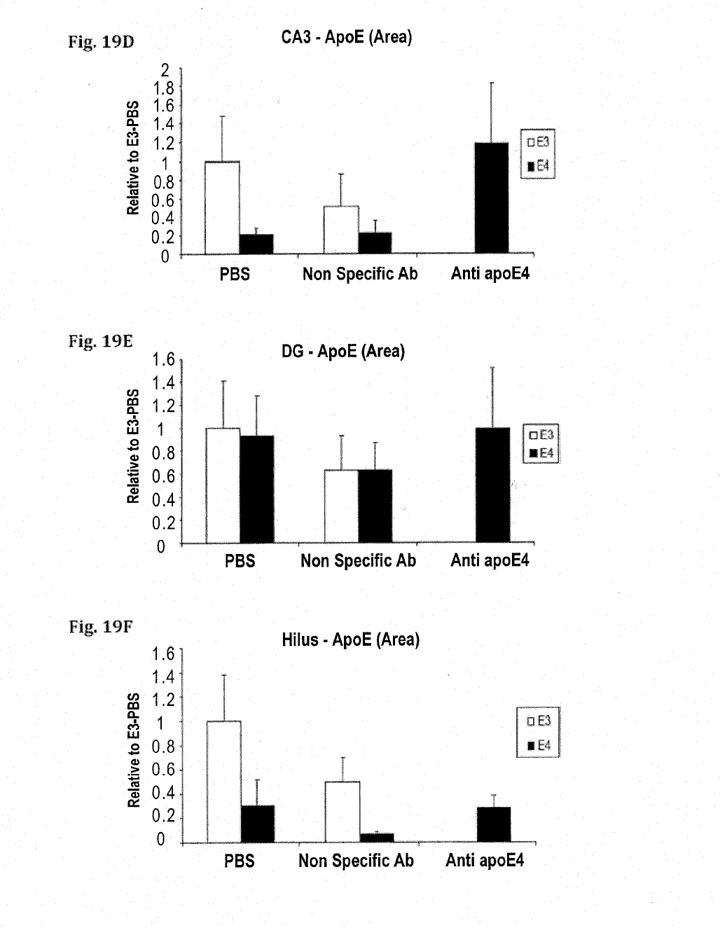

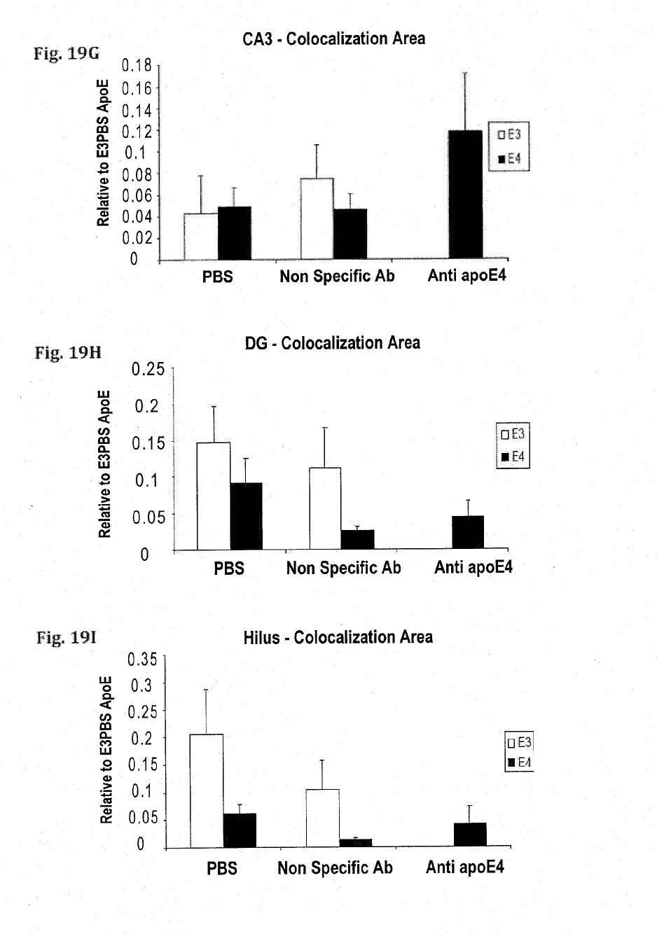

At the age of four months, the treated mice were tested by the novel object recognition analysis and the Morris water maze test. Shortly, in the novel object recognition test, mice are placed in a test arena with 2 similar objects for 5 minutes. The next day, one of the objects is replaced. The time the mouse spends near each object is measured. In the Morris water maze test, mice need to find a hidden platform in a water filled tank, over a period of 3 days (4 trials per day). Latency to platform is measured. The results of the novel object recognition test are shown in FIG. 18A and the results of the Morris maze test are shown in FIG. 18B. The results indicate that the treatment of the mice with i.p. injection of apoE4 antibody prevented the apoE4 induced decrease in the cognitive ability of the tested mice,

### Example 19

### Effects of Intraperitoneal (i.p.) Injection of apoE4 Monoclonal Antibody on IgG and hApoE Immuno-Reactivity in the Hippocampus of TR Mice

Naïve ApoE3, and apoE4 mice underwent weekly intraperitoneal (i.p.) injections of PBS, non-specific monoclonal antibody (mAb), or apoE4 monoclonal antibody (9D11) for weeks up to 4 months of age. Brain slices of the mice were subjected to immunohistochemical analysis using IgG and hApoE antibodies (n=4-6). Results presented in FIGS. 16A-C, show bar graphs of quantification of expression of IgG in CA3, DG, and Hilus of DG, respectively. Results presented in FIGS. 16D-F, show bar graphs of quantification of expression of hApoE in CA3, DG, and Hilus of DG, respectively. Results presented in FIGS. 16G-I, show bar graphs of quantification of co-localization of IgG and hApoE in CA3, DG, and Hilus of DG, respectively. The results are displayed as mean+/− SE. White bars correspond to ApoE3 mice, and black bars correspond to ApoE4 mice. * indicate P≦0.01. All results are normalized to the PBS injected ApoE3 mice. These results show that i.p. injection of apoE4 mAbs induces the accumulation of IgG and apoE4 in the hippocampus, and that a fraction of these molecules co-localize. I.p. injection of control IgG also leads to the accumulation of IgG in hippocampus but has no effect on either the levels of apoE or it's localization with apoE4.

### Example 20

### Treatment Effect of Intraperitoneal (i.p.) Injection of apoE4 Monoclonal Antibody

Three and a half months old apoE3 and apoE4 homozygote mice were i.p. injected with: PBS, non-specific (ns) IgG (MOPC-21eir, Abcam) or the apoE4 9D11 mAb (10 mg/kg). The mice were i.p. injected 3 times per week, for two weeks. The mice were sacrificed at the age of four months. Brain slices of the mice were subjected to biochemical and immunohistochemical analysis.

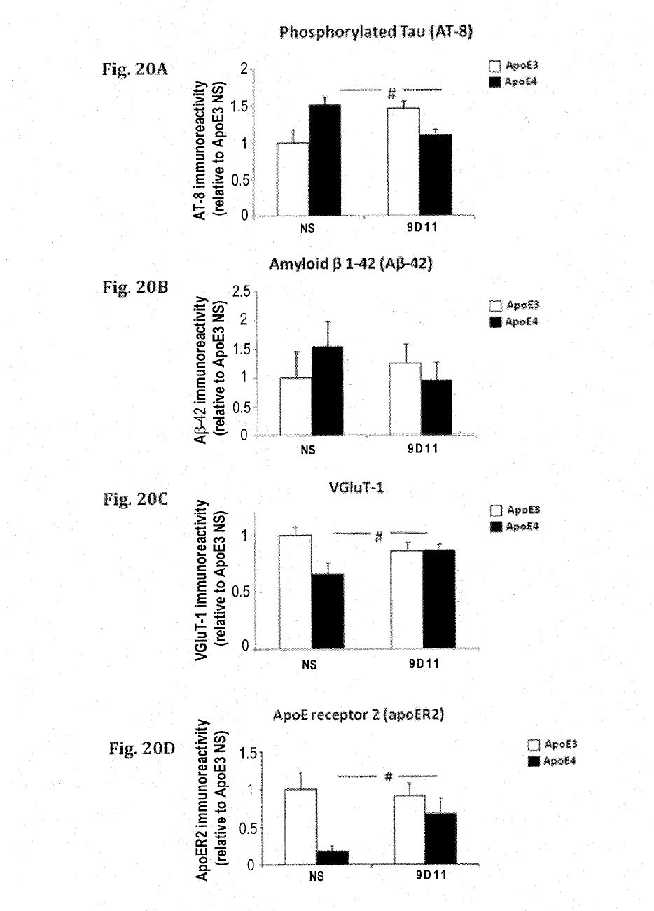

The results presented in FIGS. 20A-D show bar graphs of quantification of relative expression in the CA3 hippocampal subregion of the mice of the following markers: phosphorylated tau (AT-8, FIG. 20A), Aβ42 (Amyloid β1-42, FIG. 20B), VGluT-1 (vasicular glutamate transporter −1, FIG. 20C), and ApoE receptor 2 (apoER2, FIG. 20D). The results are displayed as mean+/− SE of relative expression compared to expression of apoE3 mice treated with non-specific antibody (NS). White bars correspond to ApoE3 mice, and black bars correspond to ApoE4 mice. NS—treatment with non-specific antibody, D9—treatment with specific apoE4 antibody. # indicates p<0.05 at a Student's T-test comparison between treatments of ApoE4.

The results indicate that i.p. treatment with apoE4 mAbs reverses the observed tau and Aβ 42 related phenotype of apoE4. In addition, the apoE4 mAbs treatment also reverses the Vglut1 and apoER2 4 phenotypes of apoE4.

All together, the surprising results presented herein, clearly show that the behavioral and brain pathological effects induced by expression of apoE4 can be counteracted by systemic administration of apoE4 antibodies. Further, the results surprisingly show that such systemic administration can be used both in preventive and therapeutic treatment protocols and regimes.

### Example 21

### Effect of Bexarotene on the Phenotype of Young apoE4 and apoE3 TR Mice

The expression of apoE is transcriptionally regulated by two ligand-activated nuclear receptors, peroxisome proliferator-activated receptor gamma (PPARγ) and liver X receptors (LXRs), which form obligate heterodimers with retinoid X receptors (RXRs). Bexarotene (Targretin) is a highly selective, blood-brain barrier-permeate RXR agonist. Oral administration of Bexarotene to a mouse model of AD resulted in enhanced apoE synthesis, and subsequently clearance of soluble Aβ within hours and reduced Aβ plaque area within just 72 hours. Furthermore, Bexarotene stimulated the rapid reversal of cognitive deficits and improved neural circuit function.

To test the effect of Bexarotene on 4-months old apoE3, apoE4 and apoE 3/4 TR mice, Bexarotene (100 mg per kg per day, (Eisai Inc. (Targretin)) was administered daily by gavage for days. The mice' brains were than excised and halved, the hippocampus was removed from one half and snap frozen for immunoblot experiments and the other half processed for immunohistochemistry.

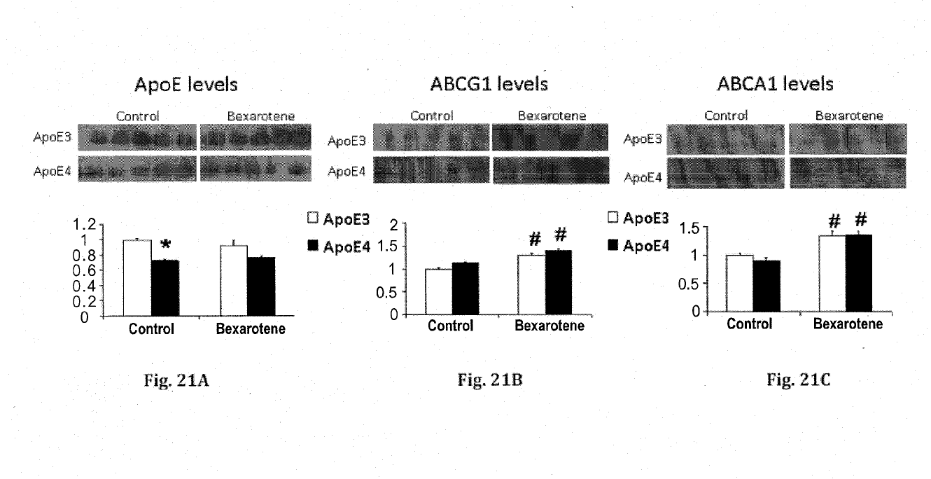

The results presented in FIG. 21A-C demonstrate the effect of Bexarotene on expression levels of apoE (FIG. 21A) and on the levels of the apoE-lapidating genes, ABCA1 (FIG. 21B) and ABCG1 (FIG. 21C), in apoE3 and apoE4 mice. The results shown in FIGS. 21A-C, are of representative western blots images (top panel) and bar graphs of quantification of the corresponding blots (lower panel) of the expression of ApoE (FIG. 21A), ABCG1 (FIG. 21B) and ABCA1 (FIG. 21C), in samples obtained from control mice or Bexarotene treated mice. The results are displayed as mean+/− SE. White bars correspond to ApoE3 mice, and black bars correspond to ApoE4 mice. *p<0.05 for differences between apoE3 and apoE4 and #p<0.05 for effects of Bexarotene.

The results show that Bexarotene increased the levels of ABCA1 (ATP binding cassette protein A1) and ABCG1 (ATP binding cassette protein G1) in both apoE3 and apoE4 mice, whereas the levels of apoE, which are lower in control apoE4 mice than in control apoE3 mice, were not affected by the treatment.

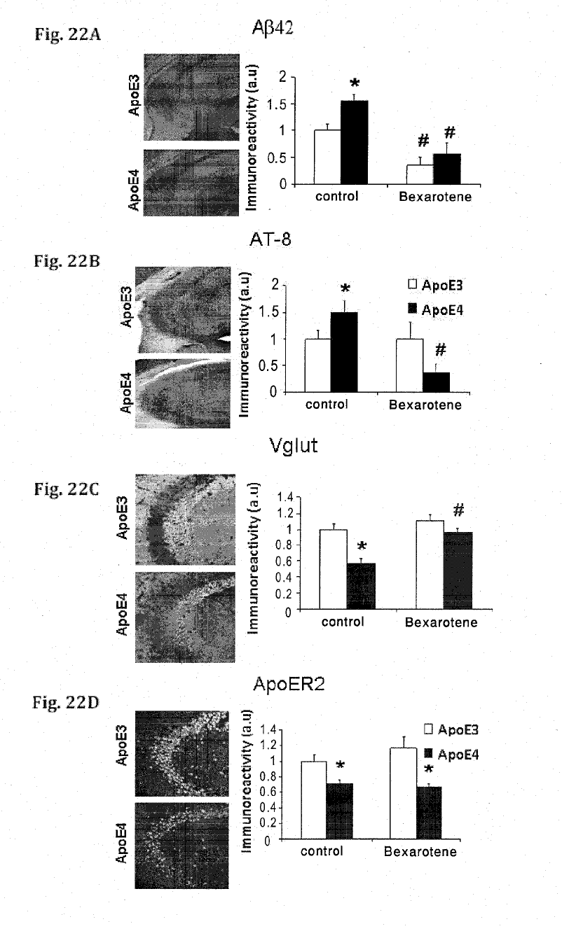

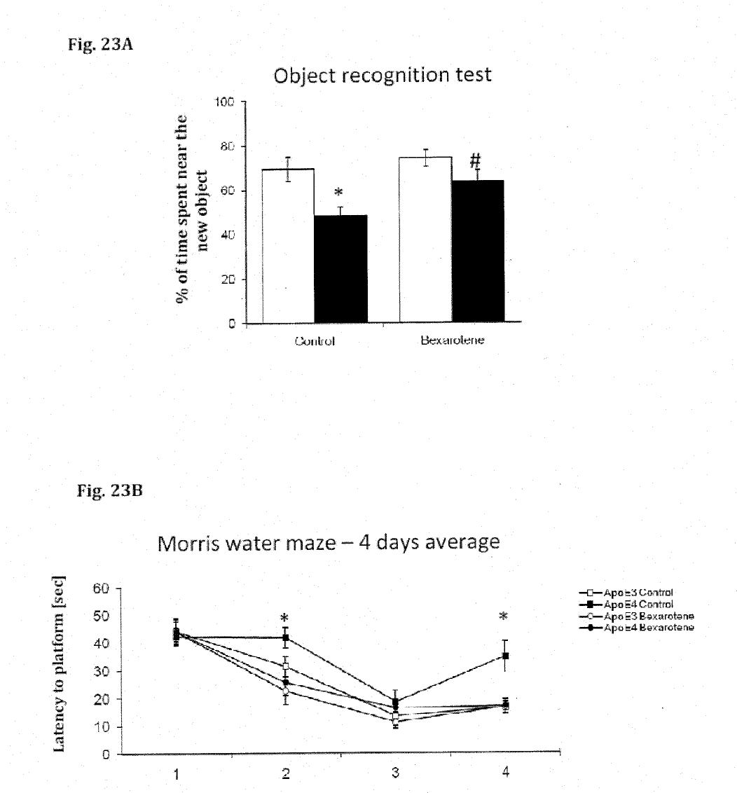

The results presented in FIGS. 22A-D demonstrate the effect of Bexarotene on accumulation of several markers which have been shown to be affected by apoE4, such as, Aβ42 expression, tau hyperphosphorylation and VGlut (i.e. synaptic improvement).

FIG. 22A shows the effect of Bexarotene on the accumulation of Aβ42. The results presented in on the left hand panel of FIG. 22A show expression of Aβ42 in representative brain sections of untreated apoE3 or ApoE4 mice. The bar graphs on the right hand panel of FIG. 22A show quantization of the results, and the difference between the control (water-treated mice) and mice treated with Bexarotene (n=6). The results are displayed as mean+/− SE. White bars correspond to ApoE3 mice, and black bars correspond to ApoE4 mice. *p<0.05 for differences between apoE3 and apoE4 and #p<0.05 for effects of Bexarotene. Altogether, the results shown in FIG. 22A demonstrate that the increased levels of Aβ42 in control apoE4 mice are abolished by Bexarotene, which reduced the levels of Aβ42 in both mouse groups.

FIG. 22B shows the effect of Bexarotene on tau hyperphosphorylation. The results presented on the left hand panel of FIG. 22B show phosphorylated tau (identified by AT-8 antibody immunostaining), in representative brain sections of untreated apoE3 or ApoE4 mice. The bar graphs on the right hand panel of FIG. 22B show quantization of the results, and the difference between the control (water-treated mice) and mice treated with Bexarotene (n=6). The results are displayed as mean+/− SE. White bars correspond to ApoE3 mice, and black bars correspond to ApoE4 mice. *p<0.05 for differences between apoE3 and apoE4 and #p<0.05 for effects of Bexarotene. Altogether, the results shown in FIG. 22B demonstrate that tau hyperphosphorylation in apoE4 control mice is reduced by Bexarotene and rendered equal to that of the apoE3 mice.

FIG. 22C shows the effect of Bexarotene on the levels of pre-synaptic marker VGlut. The results presented in on the left hand panel of FIG. 22C show Vglut expression in representative brain sections of untreated apoE3 or ApoE4 mice. The bar graphs on the right hand panel of FIG. 22C show quantization of the results, and the difference between the control (water-treated mice) and mice treated with Bexarotene (n=6). The results are displayed as mean+/− SE. White bars correspond to ApoE3 mice, and black bars correspond to ApoE4 mice. *p<0.05 for differences between apoE3 and apoE4 and #p<0.05 for effects of Bexarotene. Altogether, the results shown in FIG. 22C demonstrate that Bexarotene increased the expression levels of VGlut in the apoE4 mice, which in the control apoE4 mice are lower than the corresponding expression in the control apoE3 mice.

FIG. 22D shows the effect of Bexarotene on the levels of apoE receptor, apoER2. The results presented in on the left hand panel of FIG. 22D show apoER2 expression in representative brain sections of untreated apoE3 or ApoE4 mice. The bar graphs on the right hand panel of FIG. 22D show quantization of the results, and the difference between the control (water-treated mice) and mice treated with Bexarotene (n=6). The results are displayed as mean+/− SE. White bars correspond to ApoE3 mice, and black bars correspond to ApoE4 mice. *p<0.05 for differences between apoE3 and apoE4 and #p<0.05 for effects of Bexarotene. Altogether, the results shown in FIG. 22D, demonstrate that Bexarotene did not increase the reduced expression levels of apoER2 in apoE4 mice.

Altogether, the results presented in FIGS. 22A-D demonstrate that Bexarotene can reverse brain pathologies observed in apoE4 mice. In addition, this treatment also lowered the levels of Aβ42 in apoE3 mice to a level similar to that of the treated apoE4 mice.

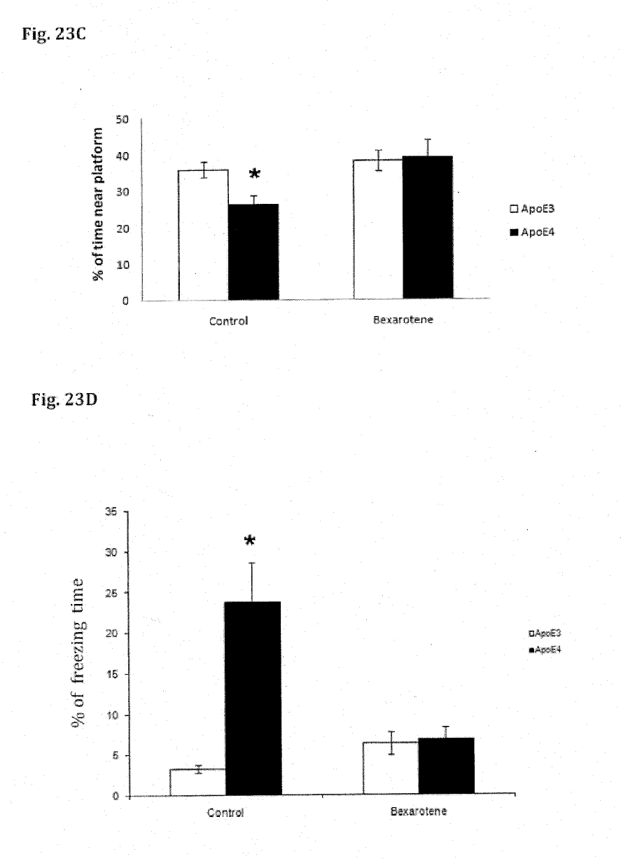

The results presented in FIGS. 23A-D demonstrate the beneficial effect of Bexarotene on apoE4 induced behavioral deficits. FIG. 23A shows the effect of Bexarotene on object recognition impairment in apoE4 mice, as measured by the object recognition test. As shown in the bar graphs represented in FIG. 23A, under control conditions, apoE4 mice (black bars) are impaired at recognizing a novel object (>=50% of the time spent near the novel object) as compared to apoE3 mice (White bars). However, in mice treated with Bexarotene the effect is reversed and the apoE4 mice prefer the novel object, similarly to the apoE3 mice. The results are displayed as mean+/− SE. White bars correspond to ApoE3 mice, and black bars correspond to ApoE4 mice. *p<0.05 for differences between apoE3 and apoE4 and #p<0.05 for effects of Bexarotene.

FIGS. 23B-C show the beneficial effect of Bexarotene on apoE4 induced behavioral deficits, as measured by the Morris swim test. As detailed above, the Morris swim test monitors the extent to which the mice learn the position of a hidden platform in a water filled arena. As seen in the line graph presented in FIG. 23B, the control apoE4 mice (E4W) take longer time (seconds, Y-Axis) to reach the platform than the control apoE3 mice (E3W), and Bexarotene treatment improves the performance of the apoE4 mice (E4B) and renders it similar to that of the apoE3 mice (E3B). The test is repeated daily over the course of four days. The results are displayed as mean+/− SE. *p<0.05 for differences between groups.

Next, the mice were further investigated following removal of the platform from the water arena. As can be seen in the bar graphs presented in FIG. 23C, the control apoE3 mice spend more time (seconds, Y-Axis) looking/searching for the missing platform in its previous location than the corresponding apoE4 mice. This difference is abolished by Bexarotene treatment, in which case both mouse groups spent the same amount of time in the relevant location. The results are displayed as mean+/− SE. White bars correspond to ApoE3 mice, and black bars correspond to ApoE4 mice. *p<0.05 for differences between groups.

FIG. 23D shows the beneficial effect of Bexarotene on stress induced by a mild electric shock in apoE4 mice (fear conditioning test). In these experiments the mice are given a small shock and the extent to which the mice freeze due to this shock is measured. The results are presented as the percentage of time the mice freeze in relation to the total amount of trial time (% of freezing time). As can be seen in the bar graphs presented in FIG. 23D, there is a significant effect of the apoE-genotype on the 1st day of the experiment, which is reversed by Bexarotene. Under control conditions, the apoE4 control mice froze for a longer time (seconds, Y-axis), as compared to the corresponding apoE3 control mice. These results are presented as percent of total time which the mice froze. Bexarotene treatment abolished this observed difference between apoE3 and apoE4 mice. The results are displayed as mean+/− SE. White bars correspond to ApoE3 mice, and black bars correspond to ApoE4 mice. *p<0.05 for differences between groups (n=7). Altogether, these results demonstrate that apoE4 mice are more stressed than the apoE3 mice in this paradigm, and that Bexarotene, in addition to counteracting the learning and memory deficits of the apoE4 mice (as demonstrated above), can also affect the elevated stress response of the apoE4 mice.

### Example 22

### Effect of Specific apoE4 Antibodies Administered in Combination with Bexarotene

3-months old apoE3, apoE4 and apoE 3/4 are i.p injected for 4 weeks (weekly injections) with apoE4 monoclonal antibody, as detailed above. These mice are orally administered with water or Bexarotene (100 mg per kg per day, Eisai Inc. (Targretin)) for 10 days prior to sacrifice, concomitantly with the i.p injections. After days, the brains are excised, halved which were each used for either biochemistry or immunohistochemistry.

### Example 23

### Intranasal Administration of apoE4 mAbs

Intranasal application of apoE4 mAbs is performed by daily administration of mg of apoE4 mAb, for 3 months starting following weaning. The delivery system is a bio-adhesive delivery system that consists of esterified hyaluronic acid microspheres. Similar experiments in which older (10 months old) mice are treated, are performed for determining the reversal effect of the treatment with apoE4 antibodies.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-40. (canceled)

41. A method of treating a neurodegenerative condition, the method comprising systemically administering to a subject in need thereof an effective amount of an antibody specific for apolipoprotein isoform apoE4 (apoE4 antibody); thereby treating the neurodegenerative condition.

42. The method according to claim 41, wherein the apoE4 antibody is a selected from a monoclonal antibody, a humanized antibody, a chimeric antibody, a single chain antibody, and antigen-binding fragments thereof.

43. The method according to claim 41, wherein the neurodegenerative condition is a neurodegenerative disease selected from Alzheimer's disease (AD), Amyotrophic Lateral Sclerosis (ALS), Primary Lateral Sclerosis (PLS), Spinal Muscular Atrophy (SMA), or any combination thereof.

44. The method according to claim 43, wherein the neurodegenerative disease is Alzheimer's disease.

45. The method according to claim 41, wherein the neurodegenerative condition is caused or induced by brain or head injury.

46. The method according to claim 45, wherein the head injury is selected from open head injury, closed brain injury and traumatic brain injury.

47. The method according to claim 41, wherein the systemic administration is carried out by a route selected from the group consisting of: intraperitoneal, intravenous, subcutaneous, intranasal, and combinations thereof.

48. The method according to claim 41, further comprising administering at least one agent capable of modulating expression of apoE.

49. The method of claim 48, wherein the at least one agent is Bexarotene.

50. A method for preventing a neurodegenerative condition, the method comprising determining the expression level of apolipoprotein isoform apoE4 in a subject; and systemically administering to a subject expressing apoE4 an effective amount of an antibody specific for apolipoprotein isoform apoE4; thereby treating the neurodegenerative condition.

51. The method according to claim 50, wherein the apoE4 antibody is a selected from a monoclonal antibody, a humanized antibody, a chimeric antibody, a single chain antibody, and antigen-binding fragments thereof.

52. The method according to claim 50, wherein the neurodegenerative condition is a neurodegenerative disease selected from Alzheimer's disease (AD), Amyotrophic Lateral Sclerosis (ALS), Primary Lateral Sclerosis (PLS), Spinal Muscular Atrophy (SMA), or any combination thereof.

53. The method according to claim 52, wherein the neurodegenerative disease is Alzheimer's disease.

54. The method according to claim 50, wherein the neurodegenerative condition is caused or induced by brain or head injury or trauma.

55. The method according to claim 50, wherein the expression is determined in a biological sample selected from a tissue or a bodily fluid.

56. The method according to claim 55, wherein the tissue is a neuronal tissue.

57. The method according to claim 50, wherein the systemic administration is carried out by a route selected from the group consisting of: intraperitoneal, intravenous, subcutaneous, intranasal, and combinations thereof.

58. The method according to claim 50, further comprising administering at least one agent capable of modulating expression and/or lipidation of apoE protein.

59. The method of claim 58, wherein the at least one agent capable of modulating expression and/or lipidation of apoE protein is administered concomitantly with the apoE4 antibody.

60. The method of claim 58, wherein the at least one agent is Bexarotene.
